(19) United States
(12) Patent Application Publication
(10) Pub. No.: US 2008/0070960 A1
(43) Pub. Date: Mar. 20, 2008

(54) Novel 3-Aryl-1,2-Benzisoxazole Derivatives and Use Thereof as Medicinal Products Against Cancer

(75) Inventors: Luc BERTIN, Crosnes (FR); Chantal CARREZ, Thiais (FR); Fabienne THOMPSON, Paris (FR); Patrick MAILLIET, Fontenay Sous Bois (FR); Jean-Marie RUXER, Issy les Moulineaux (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 11/841,128

(22) Filed: Aug. 20, 2007

Related U.S. Application Data
(63) Continuation of application No. PCT/FR2006/000375, filed on Feb. 20, 2006.

(30) Foreign Application Priority Data
Feb. 22, 2005 (FR) 0501801

(51) Int. Cl. A61K 31/423; A61P 35/00; C07D 261/20

(52) U.S. Cl. 514/379; 548/241

(57) ABSTRACT

This invention relates to 3-aryl-1,2-benzisoxazole derivatives of formula (I) 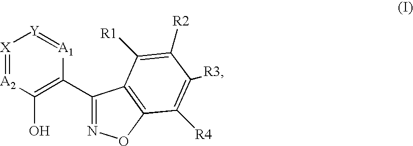 to pharmaceutical compositions comprising such derivatives, and to methods of treatment comprising administering of such derivatives.

The present invention relates to novel chemical compounds, particularly to novel 3-aryl-1,2-benzisoxazole derivatives, to compositions which contain them and to their use as medicinal products.

More particularly, according to a first aspect, the invention relates to novel benzisoxazole derivatives exhibiting an anticancer activity, and in particular an inhibitory activity on the Hsp90 chaperone protein, and even more particularly via the inhibition of the ATPase-type catalytic activity of the Hsp90 chaperone protein.

The molecular chaperones of the “Heat Shock Protein” family (HSPs), classified according to their molecular mass (Hsp27, Hsp70, Hsp90, etc.), are key elements in the balance between the synthesis and degradation of the cellular proteins responsible for correct protein folding. They play an essential role in response to cellular stress. HSPs, and in particular Hsp90, are also involved in the regulation of various major cellular functions, via their association with various client proteins involved in cell proliferation or apoptosis (Jolly C. and Morimoto R. I., J. N. Cancer Inst. (2000), 92, 1564-72; Smith D. F. et al., Pharmacological Rev. (1998), 50, 493-513; Smith D. F., Molecular Chaperones in the Cell, 165-178, Oxford University Press 2001).

Various human pathologies are the result of incorrect folding of key proteins, resulting in particular in neurodegenerative diseases subsequent to the aggregation of certain proteins, such as in Alzheimer's disease and Huntington's disease or prion-related diseases (Tytell M. and Hooper P. L., Emerging Ther. Targets (2001), 5, 3788-3796). In these pathologies, approaches aimed at upsetting or disturbing the function of chaperones could be beneficial.

The Hsp90 chaperone, which represents 1 to 2% of the protein content of the cell, has recently been demonstrated as a particularly promising target in anticancer therapy (cf. for review: Moloney A. and Workman P., Expert Opin. Biol. Ther. (2002), 2(1), 3-24; Choisis et al, Drug Discovery Today (2004), 9, 881-888). This interest comes in particular from the cytoplasmic interactions of Hsp90 with the main client proteins of Hsp90, which proteins are involved in the six mechanisms of tumour progression, as defined by Hanahan D. and Weinberg R. A. (Cell (2000), 100, 57-70), namely:
an ability to proliferate in the absence of growth factors: EGFR-R/HER2, Src, Akt, Raf, MEK, Bcr-Abl, Flt-3, etc.;
an ability to evade apoptosis: mutated form of p53, Akt, survivin, etc.;
an insensitivity to proliferation stop signals: Cdk4, Plk, Wee1, etc.;
an ability to activate angiogenesis: VEGF-R, FAK, HIF-1, Akt, etc;
an ability to proliferate without any replicative limit: hTert, etc.;
an ability to evade new tissues and to metastasize: c-Met.

Among the other client proteins for Hsp90, steroid hormone receptors, such as the oestrogen receptor or the androgen receptor, are also of considerable interest in the context of anticancer therapies.

It has recently been shown that the alpha form of Hsp90 also has an extracellular role via its interaction with the MMP-2 metalloprotease, itself involved in tumour invasion (Eustace B. K. et al, Nature Cell Biology (2004), 6, 507-514).

Hsp90 consists of two N- and C-terminal domains separated by a highly charged region. The dynamic interaction between these two domains, coordinated by the binding of nucleotides and of co-chaperones, determines the conformation of the chaperone and its activation state. The association of the client proteins depends mainly on the nature of the co-chaperones Hsp70/Hsp40, Hop60, etc., and on the nature of the ADP or ATP nucleotide bound to the N-terminal domain of Hsp 90. Thus, the hydrolysis of the ATP to ADP and the ADP/ATP exchange factor control all the chaperone “machinery”, and it has been shown that it is sufficient to prevent the hydrolysis of the ATP to ADP—ATPase activity of Hsp90—in order to release into the cytoplasm client proteins which will then be degraded in the proteasome (Neckers L and Neckers K, Expert Opin. Emerging Drugs (2002), 7, 277-288; Neckers L, Current Medicinal Chemistry, (2003), 10, 733-739; Piper P. W., Current Opin. Invest. New Drugs (2001), 2, 1606-1610).

Hsp90 Inhibitors

The first known Hsp90 inhibitors are compounds of the ansamycin family, in particular geldanamycin (1) and herbimycin A. X-ray studies have shown that geldanamycin binds to the ATP site of the N-terminal domain of Hsp90, where it inhibits the ATPase activity of the chaperone (Prodromou C. et al, Cell (1997), 90, 65-75).

Currently, the NIH and Kosan BioSciences are carrying out the clinical development of 17AAG (2), which is a geldanamycin (1)-derived Hsp90 inhibitor that blocks the ATPase activity of Hsp 90 by binding to the N-terminal ATP recognition site. The results of the phase I clinical trials for 17AAG (1) have today led to phase II trials being initiated, but also direct research towards more soluble derivatives, such as the analogue 3 (17DMAG from Kosan BioSciences), which carries a dimethylamino chain in place of the methoxy residue, and towards optimized formulations of 17AAG (CNF1010 from Conforma Therapeutics):

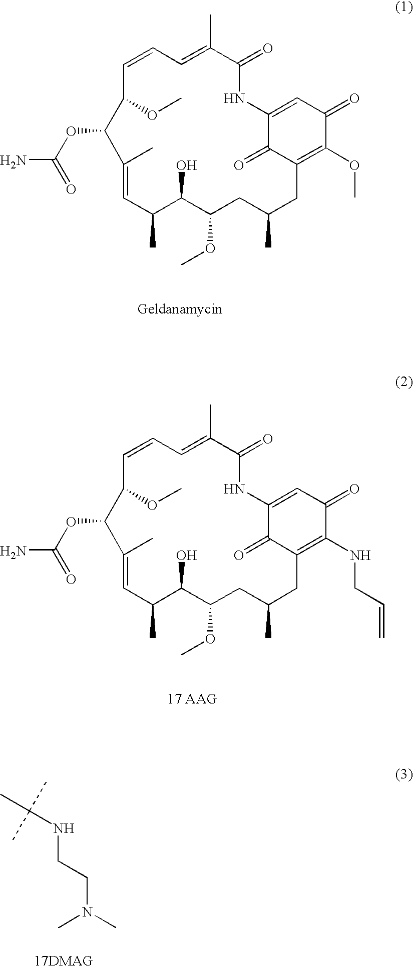

Radicicol (4) is also an Hsp 90 inhibitor of natural origin (Roe S. M. et al, J. Med. Chem. (1999), 42, 260-66). However, although the latter is by far the best in vitro Hsp90 inhibitor, its metabolic instability with respect to sulphur-containing nucleophiles makes it difficult to use in vivo. Oxime derivatives that are much more stable, such as KF 55823 (5) or KF 25706, have been developed by the company Kyowa Hakko Kogyo (Soga et al, Cancer Research (1999), 59, 2931-2938)

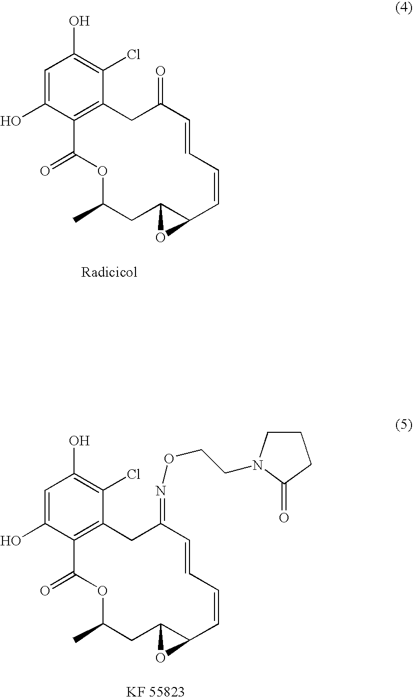

Structures of natural origin related to radicicol have also been recently described, such as zearalenone (6) by the company Conforma Therapeutics (WO 03041643) or the compounds (7-9).

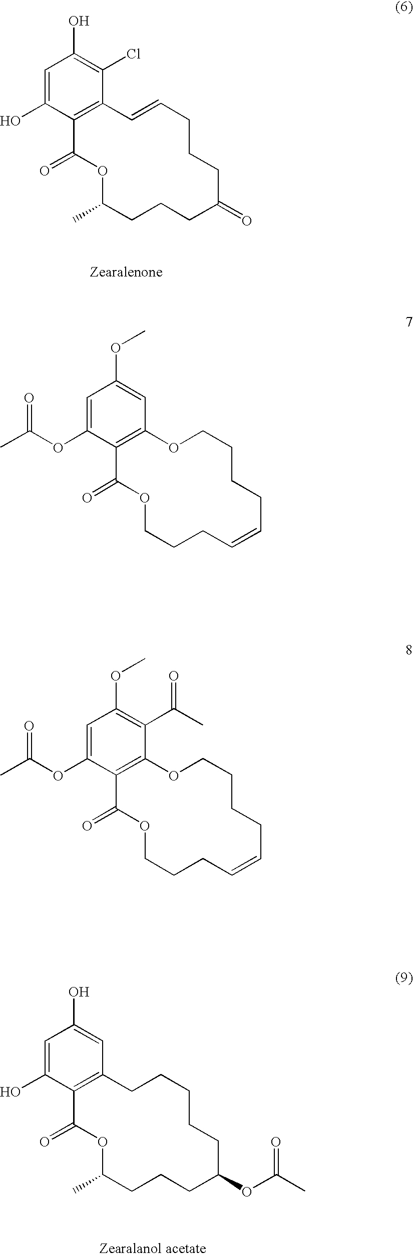

One Hsp90 inhibitor of natural origin, novobiocin (10), binds to a different ATP site located in the C-terminal domain of the protein (Itoh H. et al, Biochem J. (1999), 343, 697-703).

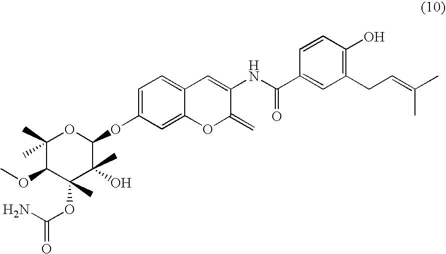

A depsipeptide, called Pipalamycin or ICI101, has just recently been described as a noncompetitive inhibitor of the ATP site of Hsp90 (J. Pharmacol. Exp. Ther. (2004), 310, 1288-1295).

Purines, such as the compounds PU3 (11) (Chiosis et al, Chem. Biol. (2001), 8, 289-299) and PU24FCI (12) (Chiosis et al, Curr. Canc. Drug Targets (2003), 3, 371-376), have also been described as Hsp90 inhibitors.

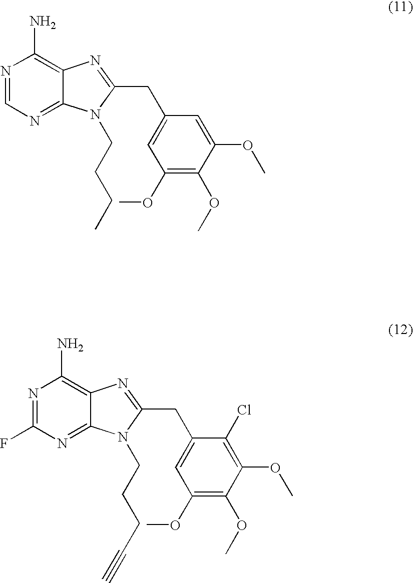

Patent application WO 2004/072080 (Cellular Genomics) claims a family of 8-heteroaryl-6-phenylimidazo[1,2-a]pyrazines as modulators of Hsp90 activity.

Patent application WO 2004/050087 (Ribotarget/Vernalis) claims a family of pyrazoles that are of use for treating pathologies related to the inhibition of heat shock proteins such as the Hsp90 chaperone.

Patent application WO 2004/056782 (Vernalis) claims a novel family of pyrazoles that are of use for treating pathologies related to the inhibition of heat shock proteins such as the Hsp90 chaperone.

Patent application WO 2004/072051 (Vernalis) claims arylisoxazole derivatives that are of use for treating pathologies related to the inhibition of heat shock proteins such as the Hsp90 chaperone.

Patent application WO 2004/096212 (Vernalis) claims a third family of pyrazoles that are of use for treating pathologies related to the inhibition of heat shock proteins such as the Hsp90 chaperone.

Patent application WO 2005/00300 (Vernalis) claims, more generally, heterocycles with 5 ring members, substituted with aryl radicals, that are of use for treating pathologies related to the inhibition of heat shock proteins such as the Hsp90 chaperone.

Finally, patent application WO 2005/00778 (Kyowa Hakko Kogyo) claims a family of benzophenone derivatives as Hsp90 inhibitors, that are of use for the treatment of tumours.

The present invention thus relates to 3-aryl-1,2-benzisoxazole derivatives corresponding to the following formula (I): 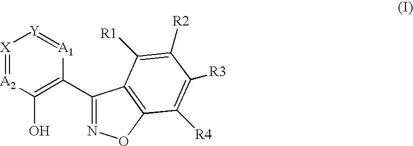
in which:
A1 and A2, which may be identical or different, represent CH or N;
X represents CRa or N;
Y represents CRb or N;
R₁, R₂, R₃ and R₄, which may be identical or different, are chosen from hydrogen or halogen atoms, or cyano, nitro, trifluoromethyl, OR₅, SR₅, NR₅R₆, C(O)R₅, C(O)OR₅, C(O)NR₅R₆, S(O)R₅, S(O)₂R₅, S(O)NR₅R₆, S(O)₂NR₅R₆, NR₆C(O)R₅, NR₆C(O)OR₅, alkyl, alkenyl, alkynyl, aryl, heteroaryl, aralkyl or heteroaralkyl radicals; it being understood that the alkyl, alkenyl, alkynyl, aryl, heteroaryl, aralkyl or heteroaralkyl radicals can be substituted;
Ra represents a halogen atom, a hydroxyl radical or a nitro radical when Rb is a hydrogen atom; Ra represents a hydrogen or a halogen atom, or a hydroxyl, methyl, ethyl, alkoxy, hydroxymethyl, nitro, carboxyl or cyano radical when Rb is different from a hydrogen atom;
Rb represents a hydrogen or halogen atom, a cyano, nitro or trifluoromethyl radical or a (C₁-C₃ alkyl)n-W—(C₁-C₃ alkyl)m-R₅ radical;
R₅ and R₆ are independently chosen from a hydrogen atom or alkyl, alkenyl, alkynyl, aryl, heteroaryl, aralkyl or heteroaralkyl radicals; it being understood that the alkyl, alkenyl, alkynyl, aryl, heteroaryl, aralkyl or heteroaralkyl radicals can be substituted;
n and m are independently chosen from 0 and 1;
W is chosen from the group formed by a single bond, oxygen or sulphur atoms, and C₁-C₃ alkyl, C₁-C₃ alkenyl, C₁-C₃ alkynyl, C₃-C₇ cycloalkyl, C₃-C₇ heterocycloalkyl, aryl, heteroaryl, NH, —CH═N—, N—(C₁-C₃ alkyl), C(O), C(O)—O, C(O)—NH, C(O)—N(C₁-C₃ alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃ alkyl), NH—S(O), NH—S(O)₂, N(C₁-C₃ alkyl)-S(O), N(C₁-C₃ alkyl)-S(O)₂, O—S(O), O—S(O)₂, S(O)—O, S(O)₂—O, O—P(C₁-C₃ alkyl)(O), O—P(O)₂, NH—P(C₁-C₃ alkyl)(O), NH—P(O)₂, P(C₁-C₃ alkyl)(O)—NH or P(O)₂—NH radicals,
   with the exclusion of the following product: 4-(1,2-benzisoxazol-3-yl)benzene-1,3-diol,
   said products of formula (I) being in any of the possible racemic, enantiomeric and diastereoisomer isomer forms, and also the addition salts with inorganic and organic acids or with inorganic and organic bases of said products of formula (I).

The present invention thus relates to 3-aryl-1,2-benzisoxazole derivatives corresponding to the following formula (I): 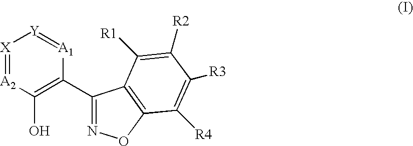
in which:
A1 and A2, which may be identical or different, represent CH or N;
X represents CRa or N;
Y represents CRb or N;
R₁, R₂, R₃ and R₄, which may be identical or different, are chosen from hydrogen or halogen atoms, or cyano, nitro, trifluoromethyl, OR₅, SR₅, NR₅R₆, C(O)R₅, C(O)OR₅, C(O)NR₅R₆, S(O)R₅, S(O)₂R₅, S(O)NR₅R₆, S(O)₂NR₅R₆, NR₆C(O)R₅, NR₆C(O)OR₅, alkyl, alkenyl, alkynyl, aryl, heteroaryl, aralkyl or heteroaralkyl radicals; it being understood that the alkyl, alkenyl, alkynyl, aryl, heteroaryl, aralkyl or heteroaralkyl radicals can be substituted;
Ra represents a halogen atom, a hydroxyl radical or a nitro radical when Rb is a hydrogen atom; Ra represents a hydrogen or a halogen atom, or a hydroxyl, methyl, ethyl, methoxy, hydroxymethyl, nitro, carboxyl or cyano radical when Rb is different from a hydrogen atom;
Rb represents a hydrogen or halogen atom, a cyano, nitro or trifluoromethyl radical or a (C₁-C₃ alkyl)n-W—(C₁-C₃ alkyl)m-R₅ radical;
R₅ and R₆ are independently chosen from a hydrogen atom or alkyl, alkenyl, alkynyl, aryl, heteroaryl, aralkyl or heteroaralkyl radicals; it being understood that the alkyl, alkenyl, alkynyl, aryl, heteroaryl, aralkyl or heteroaralkyl radicals can be substituted;
n and m are independently chosen from 0 and 1;
W is chosen from the group formed by a single bond, oxygen or sulphur atoms, and C₁-C₃ alkyl, C₂-C₃ alkenyl, C₂-C₃ alkynyl, C₃-C₇ cycloalkyl, C₃-C₇ heterocycloalkyl, aryl, heteroaryl, NH, N—(C₁-C₃ alkyl), C(O), C(O)—O, C(O)—NH, C(O)—N(C₁-C₃ alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃ alkyl), NH—S(O), NH—S(O)₂, N(C₁-C₃ alkyl)-S(O), N(C₁-C₃ alkyl)-S(O)₂, O—S(O), O—S(O)₂, S(O)—O, S(O)₂—O, O—P(C₁-C₃ alkyl)(O), O—P(O)₂, NH—P(C₁-C₃ alkyl)(O), NH—P(O)₂, P(C₁-C₃ alkyl)(O)—NH or P(O)₂—NH radicals;
   said products of formula (I) being in any of the possible racemic, enantiomeric and diastereoisomeric isomer forms, and also the addition salts with inorganic and organic acids or with inorganic and organic bases of said products of formula (I).

The present invention does not relate to the products of formula (I) in which:
1. either any one of the radicals R₁, R₂, R₃ and R₄ represents a hydroxyl radical; such compounds being described, as antioestrogenic agents with antineoplastic activity, in patent US 2003 207927, or as ligands selective for the ERβ oestrogen receptor in J. Med. Chem. 2004, 47(21), 5021-40;
2. or the radical R₃ represents a 4(3H)-pyrimidinone or thione derivative; such compounds being described, as herbicides, in various patents, including EP 908457;
3. or the radical R₃ represents a -Z-CRxRy-COOH group, with Z=O or S, and Rx, Ry=H, F, alkyl, alkenyl, alkynyl, haloalkyl, haloalkenyl or haloalkynyl, or else CRxRy=cycloalkyl; such compounds being described, as agents used in the treatment of diabetes and of other lipid disorders, in patent US 2002 173663.

A synthesis of the compound [86013-74-3] was described in J. Org. Chem. 1983, 48(15), 2613-15, without the mention of any biological activity. A synthesis of the compound [173736-14-6] was described as an intermediate product used in the preparation of herbicides in U.S. Pat. No. 5,484,763, without the mention of any biological activity. Finally, a synthesis of the compound [78578-95-2] was described in J. Med. Chem. 2004, 47(21), 5021-40, where it is mentioned that this product behaves as a selective ligand for the ERβ oestrogen receptor.

| 86013-74-3 | 173736-14-6 | 78578-95-2 |
|---|---|---|
| 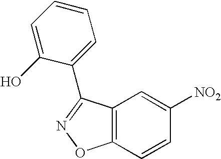 | 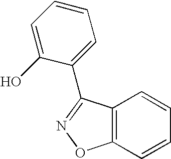 | 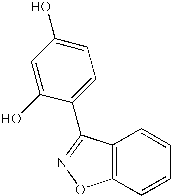 |

In the products of formula (I) and in the subsequent text, the terms indicated have the following meanings:
The term “halogen” denotes fluorine, chlorine, bromine or iodine atoms, and preferably chlorine or bromine atoms.
The term “alkyl radical” denotes a linear or branched radical containing at most 12 carbon atoms, chosen from methyl, ethyl, propyl, isopropyl, butyl, isobutyl, sec-butyl, tert-butyl, pentyl, isopentyl, sec-pentyl, tert-pentyl, neopentyl, hexyl, isohexyl, sec-hexyl, tert-hexyl and also heptyl, octyl, nonyl, decyl, undecyl and dodecyl radicals, and also the linear or branched positional isomers thereof. Mention is more particularly made of alkyl radicals having at most 6 carbon atoms, and in particular methyl, ethyl, propyl, isopropyl, n-butyl, isobutyl, tert-butyl, linear or branched pentyl, or linear or branched hexyl radicals.
The term “alkenyl radical” denotes a linear or branched radical containing at most 12 carbon atoms, and preferably 4 carbon atoms, chosen, for example, from the following values: ethenyl or vinyl, propenyl or allyl, 1-propenyl, n-butenyl, i-butenyl, 3-methylbut-2-enyl, n-pentenyl, hexenyl, heptenyl, octenyl, cyclohexylbutenyl and decenyl, and also the linear or branched positional isomers thereof. Among the alkenyl values, mention is more particularly made of the allyl or butenyl values.
The term “alkynyl radical” denotes a linear or branched radical containing at most 12 carbon atoms, and preferably 4 carbon atoms, chosen, for example, from the following values: ethynyl, propynyl or propargyl, butynyl, n-butynyl, i-butynyl, 3-methylbut-2-ynyl, pentynyl or hexynyl, and also the linear or branched positional isomers thereof. Among the alkynyl values, mention is more particularly made of the propargyl value.
The term “alkoxy radical”, which can represent, for example, OR5, denotes a linear or branched radical containing at most 12 carbon atoms, and preferably 6 carbon atoms, chosen, for example, from methoxy, ethoxy, propoxy, isopropoxy, linear butoxy, which may be secondary or tertiary, pentoxy, hexoxy and heptoxy radicals, and also the linear or branched positional isomers thereof.
The term “alkylthio” or “alkyl-S-”, which can represent, for example, SR5, denotes a linear or branched radical containing at most 12 carbon atoms and represents in particular methylthio, ethylthio, isopropylthio and heptylthio radicals. In the radicals containing a sulphur atom, the sulphur atom may be oxidized to an SO or S(O)₂ radical.
The term “acyl radical” or “R—CO—” denotes a linear or branched radical containing at most 12 carbon atoms, in which the radical r represents a hydrogen atom, or an alkyl, cycloalkyl, cycloalkenyl, cycloalkyl, heterocycloalkyl or aryl radical, these radicals having the values indicated above and being optionally substituted as indicated: mention is, for example, made of formyl, acetyl, propionyl, butyryl or benzoyl, or else valeryl, hexanoyl, acryloyl, crotonoyl or carbamoyl radicals.
The term “cycloalkyl radical” denotes a monocyclic or bicyclic carbocyclic radical containing from 3 to 10 ring members and denotes in particular cyclopropyl, cyclobutyl, cyclopentyl and cyclohexyl radicals.
The term “cycloalkylalkyl” radical denotes a radical in which the cycloalkyl and alkyl are chosen from the values indicated above: this radical thus denotes, for example, cyclopropylmethyl, cyclopentylmethyl, cyclohexylmethyl and cycloheptylmethyl radicals.
The term “acyloxy radical” is intended to mean acyl-O— radicals in which acyl has the meaning indicated above: mention is, for example, made of acetoxy or propionyloxy radicals.
The term “acylamino radical” is intended to mean acyl-N— radicals in which acyl has the meaning indicated above.
The term “aryl radical” denotes unsaturated carbocyclic radicals that are monocyclic or consist of condensed rings. As examples of such an aryl radical, mention may be made of phenyl or naphthyl radicals: mention is more particularly made of the phenyl radical.
The term “arylalkyl” is intended to mean radicals resulting from the combination of the alkyl radicals mentioned above that are optionally substituted and the aryl radicals also mentioned above, that are optionally substituted: mention is, for example, made of benzyl, phenylethyl, 2-phenethyl, triphenylmethyl or naphthalenemethyl radicals.
The term “heterocyclic radical” denotes a saturated (heterocycloalkyl) or unsaturated (heteroaryl) carbocyclic radical consisting at most of 6 ring members interrupted with one or more hetero atoms, which may be identical or different, chosen from oxygen, nitrogen or sulphur atoms.

As heterocycloalkyl radicals, mention may in particular be made of dioxolane, dioxane, dithiolane, thiooxolane, thiooxane, oxiranyl, oxolanyl, dioxolanyl, piperazinyl, piperidinyl, pyrrolidinyl, imidazolidinyl, pyrazolidinyl, morpholinyl or else tetrahydrofuryl, tetrahydrothienyl, chromanyl, dihydrobenzofuranyl, indolinyl, piperidinyl, perhydropyranyl, pyrindolinyl, tetrahydroquinolinyl, tetrahydroisoquinolinyl or thioazolidinyl radicals, all these radicals being optionally substituted.

Among the heterocycloalkyl radicals, mention may in particular be made of optionally substituted piperazinyl, optionally substituted piperidinyl, optionally substituted pyrrolidinyl, imidazolidinyl, pyrazolidinyl, morpholinyl or thioazolidinyl radicals.
The term “heterocycloalkylalkyl radical” is intended to mean radicals in which the heterocycloalkyl and alkyl residues have the meanings above.
Among the heteroaryl radicals with 5 ring members, mention may be made of furyl, such as 2-furyl, thienyl, such as 2-thienyl and 3-thienyl, pyrrolyl, diazolyl, thiazolyl, thiadiazolyl, thiatriazolyl, isothiazolyl, oxazolyl oxadiazolyl, 3- or 4-isoxazolyl, imidazolyl, pyrazolyl and isoxazolyl radicals.
Among the heteroaryl radicals with 6 ring members, mention may in particular be made of pyridyl, such as 2-pyridyl, 3-pyridyl and 4-pyridyl, pyrimidyl, pyrimidinyl, pyridazinyl, pyrazinyl and tetrazolyl radicals.
As condensed heteroaryl radicals containing at least one hetero atom chosen from sulphur, nitrogen and oxygen, mention may, for example, be made of benzothienyl, such as 3-benzothienyl, benzofuryl, benzofuranyl, benzopyrrolyl, benzimidazolyl, benzoxazolyl, thionaphthyl, indolyl, purinyl, quinolinyl, isoquinolinyl and naphthyridinyl.

Among the condensed heteroaryl radicals, mention may more particularly be made of benzothienyl, benzofuranyl, indolyl, quinolinyl, benzimidazolyl, benzothiazolyl, furyl, imidazolyl, indolizinyl, isoxazolyl, isoquinolinyl, isothiazolyl, oxadiazolyl, pyrazinyl, pyridazinyl, pyrazolyl, pyridyl, pyrimidinyl, pyrrolyl, quinazolinyl, 1,3,4-thiadiazolyl, thiazolyl and thienyl radicals and triazolyl groups, these radicals being optionally substituted as indicated for the heteroaryl radicals.
The term “cyclic amine”, which can represent, for example, NR5R6, denotes a cycloalkyl radical containing from 3 to 8 ring members, in which a carbon atom is replaced with a nitrogen atom, the cycloalkyl radical having the meaning indicated above and possibly also containing one or more other hetero atoms chosen from O, S, SO₂, N or NR7 with R7 as defined above; as examples of such cyclic amines, mention may, for example, be made of pyrrolidinyl, piperidinyl, morpholinyl, piperazinyl, indolinyl, pyrindolinyl or tetrahydroquinolinyl radicals.
The term “patient” denotes human beings, but also the other mammals.
The term “prodrug” denotes a product which can be converted in vivo by metabolic mechanisms (such as hydrolysis) into a product of formula (I). For example, an ester of a product of formula (I) containing a hydroxyl group can be converted, by hydrolysis in vivo, into its parent molecule. Alternatively, an ester of a product of formula (I) containing a carboxyl group can be converted, by hydrolysis in vivo, into its parent molecule.

By way of example, mention may be made of esters of products of formula (I) containing a hydroxyl group, such as acetates, citrates, lactates, tartrates, malonates, oxalates, salicylates, propionates, succinates, fumarates, maleates, methylenebis-b-hydroxynaphthoates, gentisates, isethionates, di-p-tolyltartrates, methanesulphonates, ethanesulphonates, benzenesulphonates, p-toluenesulphonates, cyclohexylsulphamates and quinates.

Esters of products of formula (I) which are particularly useful, containing a hydroxyl group, can be prepared from acid residues such as those described by Bundgaard et. al., J. Med. Chem., 1989, 32, page 2503-2507: these esters include in particular substituted (aminomethyl)benzoates, dialkylamino-methylbenzoates in which the two alkyl groups may be linked together or may be interrupted with an oxygen atom or with a nitrogen atom that is optionally substituted, i.e. an alkylated nitrogen atom, or alternatively (morpholino-methyl)benzoates, e.g. 3- or 4-(morpholinomethyl)benzoates, and (4-alkyl-piperazin-1-yl)benzoates, e.g. 3- or 4-(4-alkylpiperazin-1-yl)benzoates.

The carboxyl radical(s) of the products of formula (I) may be salified or esterified with various groups known to those skilled in the art, among which mention may be made, by way of nonlimiting examples, of the following compounds:
among the salification compounds, inorganic bases such as, for example, an equivalent of sodium, potassium, lithium, calcium, magnesium or of ammonium or organic bases such as, for example, methylamine, propylamine, trimethylamine, diethylamine, triethylamine, N,N-dimethyl-ethanolamine, tris(hydroxymethyl)aminomethane, ethanolamine, pyridine, picoline, dicyclohexylamine, morpholine, benzylamine, procane, lysine, arginine, histidine or N-methylglucamine,
among the esterification compounds, alkyl radicals to form alkoxycarbonyl groups, such as, for example, methoxycarbonyl, ethoxycarbonyl, tert-butoxy-carbonyl or benzyloxycarbonyl, these radicals possibly being substituted with radicals chosen, for example, from halogen atoms, and hydroxyl, alkoxy, acyl, acyloxy, alkylthio, amino or aryl radicals, such as, for example, in chloromethyl, hydroxypropyl, methoxymethyl, propionyloxymethyl, methylthiomethyl, dimethylaminoethyl, benzyl or phenethyl groups.

The term “esterified carboxyl” is intended to mean, for example, radicals such as alkyloxycarbonyl radicals, for example methoxycarbonyl, ethoxycarbonyl, propoxycarbonyl, butyl or tert-butyloxycarbonyl, cyclobutyloxycarbonyl, cyclopentyloxycarbonyl or cyclohexyloxycarbonyl.

Mention may also be made of radicals formed with readily cleavable ester residues, such as methoxymethyl or ethoxymethyl radicals; acyloxyalkyl radicals such as pivaloyloxymethyl, pivaloyloxyethyl, acetoxymethyl or acetoxyethyl; alkyloxycarbonyloxy alkyl radicals such as methoxycarbonyloxy methyl or ethyl radicals, and isopropyloxycarbonyloxy methyl or ethyl radicals.

A list of such ester radicals can be found, for example, in European patent EP 0 034 536.

The term “amidated carboxyl” is intended to mean, for example, radicals of the type —CONR5R6 in which R5 and R6 have the meanings indicated above.

The term “alkylamino radical” is intended to mean radicals in which the alkyl radical is chosen from the alkyl radicals mentioned above. Preference is given to alkyl radicals having at most 4 carbon atoms and mention may, for example, be made of methylamino radicals, ethylamino radicals, propylamino radicals or butylamino radicals, which may be linear or branched.

The term “dialkylamino radical” is intended to mean radicals in which the alkyl radicals, which may be identical or different, are chosen from the alkyl radicals mentioned above. As previously, preference is given to alkyl radicals having at most 4 carbon atoms and mention may, for example, be made of dimethylamino radicals, diethylamino radicals, or methylethylamino radicals, which may be linear or branched.

The NR5R6 radicals may also represent a heterocycle, which may or may not comprise an additional hetero atom. Mention may be made of pyrrolyl, imidazolyl, indolyl, piperidinyl, morpholinyl and piperazinyl radicals. Piperidinyl, morpholinyl or piperazinyl radicals are preferred.

The term “salified carboxyl” is intended to mean the salts formed, for example, with one equivalent of sodium, of potassium, of lithium, of calcium, of magnesium or of ammonium. Mention may also be made of the salts formed with organic bases such as methylamine, propylamine, trimethylamine, diethylamine or triethylamine. The sodium salt is preferred.

When the products of formula (I) comprise an amino radical that can be salified with an acid, it is clearly understood that these acid salts also form part of the invention. Mention may be made of the salts obtained, for example, with hydrochloric acid or methanesulphonic acid.

The addition salts with inorganic or organic acids of the products of formula (I) may, for example, be the salts formed with hydrochloric acid, hydrobromic acid, hydriodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulphonic acids such as, for example, methanesulphonic acid, ethanesulphonic acid or propanesulphonic acid, alkyldisulphonic acids such as, for example, methanedisulphonic acid or alpha, beta-ethanedisulphonic acid, arylmonosulphonic acids such as benzenesulphonic acid, and aryldisulphonic acids.

It may be recalled that the stereoisomerism may be defined in its broad sense as the isomerism of compounds having the same structural formulae but whose various groups are arranged differently in space, such as in particular in monosubstituted cyclohexanes whose substituent may be in the axial or equatorial position, and the various possibly rotational conformations of ethane derivatives. However, there is another type of stereoisomerism, due to the different spatial arrangements of fixed substituents, either on double bonds or on rings, which is often referred to as geometrical isomerism or cis-trans isomerism. The term “stereoisomer” is used in the present application in its broadest sense and thus relates to all the compounds indicated above.

The present invention thus relates in particular to the products of formula (I) as defined above in which
A1 and A2, which may be identical or different, represent CH or N;
X represents CRa or N;
Y represents CRb or N;
R₁, R₂, R₃ and R₄, which may be identical or different, are chosen from hydrogen or halogen atoms, or cyano, nitro, trifluoromethyl, OR₅, SR₅, NR₅R₆, C(O)R₅, C(O)OR₅, C(O)NR₅R₆, S(O)₂NR₅R₆, NR₆C(O)R₅, NR₆C(O)OR₅, alkyl, aryl, heteroaryl, aralkyl or heteroaralkyl radicals; it being understood that the alkyl, alkenyl, alkynyl, aryl, heteroaryl, aralkyl or heteroaralkyl radicals are optionally substituted;
Ra represents a halogen atom, a hydroxyl or alkoxy radical or a nitro radical;
Rb represents a hydrogen or halogen atom or a (C₁-C₃ alkyl)n-W—(C₁-C₃ alkyl)m-R5 radical;
R5 and R6 are independently chosen from a hydrogen atom or alkyl, alkenyl, alkynyl, aryl, heteroaryl, aralkyl or heteroaralkyl radicals; it being understood that the alkyl, alkenyl, alkynyl, aryl, heteroaryl, aralkyl or heteroaralkyl radicals are optionally substituted;
n and m are independently chosen from 0 and 1;
W is chosen from the group formed by a single bond, oxygen atoms, and C₁-C₃ alkyl, C₂-C₃ alkenyl, C₃-C₇ heterocycloalkyl, aryl, heteroaryl, NH, —CH═N—, N—(C₁-C₃alkyl), C(O), C(O)—O, C(O)—NH, C(O)—N(C₁-C₃alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃ alkyl), NH—S(O), NH—S(O)₂, N(C₁-C₃ alkyl)-S(O), N(C₁-C₃ alkyl)-S(O)₂, O—S(O), O—S(O)₂, S(O)—O or S(O)₂—O radicals;
   said products of formula (I) being in any of the possible racemic, enantiomeric and diastereoisomeric isomer forms, and also the addition salts with inorganic and organic acids or with inorganic and organic bases of said products of formula (I).

The present invention thus relates in particular to the products of formula (I) as defined above in which
A1 and A2, which may be identical or different, represent CH or N;
X represents CRa or N;
Y represents CRb or N;
R₁, R₂, R₃ and R₄, which may be identical or different, are chosen from hydrogen or halogen atoms, or cyano, nitro, trifluoromethyl, OR₅, SR₅, NR₅R₆, C(O)R₅, C(O)OR₅, C(O)NR₅R₆, S(O)₂NR₅R₆, NR₆C(O)R₅, NR₆C(O)OR₅, alkyl, aryl, heteroaryl, aralkyl or heteroaralkyl radicals; it being understood that the alkyl, alkenyl, alkynyl, aryl, heteroaryl, aralkyl or heteroaralkyl radicals are optionally substituted;
Ra represents a halogen atom, a hydroxyl radical or a nitro radical;
Rb represents a hydrogen or halogen atom or a (C₁-C₃ alkyl)n-W—(C₁-C₃ alkyl)m-R₅ radical;
R₅ and R₆ are independently chosen from a hydrogen atom or alkyl, alkenyl, alkynyl, aryl, heteroaryl, aralkyl or heteroaralkyl radicals; it being understood that the alkyl, alkenyl, alkynyl, aryl, heteroaryl, aralkyl or heteroaralkyl radicals are optionally substituted;
n and m are independently chosen from 0 and 1;
W is chosen form the group formed by a single bond, oxygen atoms, and C₁-C₃ alkyl, C₂-C₃ alkenyl, C₃-C₇ heterocycloalkyl, aryl, heteroaryl, NH, N—(C₁-C₃ alkyl), C(O), C(O)—O, C(O)—NH, C(O)—N(C₁-C₃ alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃ alkyl), NH—S(O), NH—S(O)₂, N(C₁-C₃ alkyl)-S(O), N(C₁-C₃ alkyl)-S(O)₂, O—S(O), O—S(O)₂, S(O)—O or S(O)₂—O radicals;
   said products of formula (I) being in any of the possible racemic, enantiomeric and diastereoisomeric isomer forms, and also the addition salts with inorganic and organic acids or with inorganic and organic bases of said products of formula (I).

The present invention thus relates in particular to the products of formula (I) as defined above in which
A1 and A2, which may be identical or different, represent CH or N;
X represents CRa or N;
Y represents CRb or N;
R₁, R₂, R₃ and R₄, which may be identical or different, are chosen from hydrogen and halogen atoms and —OR₅ radicals;
Ra represents a halogen atom or a hydroxyl or alkoxy radical;
Rb represents a hydrogen or halogen atom or a (C₁-C₃ alkyl)n-W—R₅ radical;
R5 is chosen from alkyl, aralkyl, aryl or heteroaralkyl radicals, all optionally substituted;
W represents a single bond, C(O), C(O)—O or C(O)—NH; NH or —CH═N—, n represents 0 or 1;
   said products of formula (I) being in any of the possible racemic, enantiomeric and diastereoisomeric isomer forms, and also the addition salts with inorganic and organic acids or with inorganic and organic bases of said products of formula (I).

The present invention thus relates in particular to the products of formula (I) as defined above in which
A1 and A2, which may be identical or different, represent CH or N;
X represents CRa or N;
Y represents CRb or N;
R₁, R₂, R₃ et R₄, which may be identical or different, are chosen from hydrogen and halogen atoms and —OR₅ radicals;
Ra represents a halogen atom or a hydroxyl radical;
Rb represents a hydrogen or halogen atom or a W—R₅ radical;
R₅ is chosen from alkyl, aralkyl or heteroaralkyl radicals, all optionally substituted;
W represents C(O), C(O)—O or C(O)—NH; said products of formula (I) being in any of the possible racemic, enantiomeric and diastereoisomeric isomer forms, and also the addition salts with inorganic and organic acids or with inorganic and organic bases of said products of formula (I).

The present invention thus relates in particular to the products of formula (I) as defined above in which A1 and A2 represent CH, the other substituents X, Y, R1, R2, R3 and R4 of said products of formula (I) having the values defined in any one of the preceding claims,

said products of formula (I) being in any of the possible racemic, enantiomeric and diastereoisomeric isomer forms, and also the addition salts with inorganic and organic acids or with inorganic and organic bases of said products of formula (I).

In the products of formula (I) as defined above and below, the alkyl, alkenyl, alkynyl, aryl, heteroaryl, aralkyl or heteroaralkyl radicals can be optionally substituted with one or more radicals, which may be identical or different, chosen from halogen atoms; and the radicals: hydroxyl; cycloalkyl containing at most 6 ring members; acyl containing at most 7 carbon atoms; cyano; nitro; free, salified or esterified carboxyl; tetrazolyl; —NH₂, —NH(alk), —N(alk)(alk); SO₂—NH—CO—NH-alkyl; SO₂—NH—CO—NH-phenyl; —C(O)—NH₂; —C(O)—NH(alk); —C(O)—N(alk)(alk), —NH—C(O)-(alk), —N(alk)-C(O)-(alk); thienyl; phenyl, alkyl, alkylthio, alkoxy and phenoxy, themselves optionally substituted with one or more radicals chosen from halogen atoms and hydroxyl, alkoxy, alkyl, —NH₂, —NH(alk) and —N(alk)(alk) radicals.

More particularly, in the products of formula (I) as defined above and below, the alkyl, alkenyl, alkynyl, aryl, heteroaryl, aralkyl or heteroaralkyl radicals can be optionally substituted with one or more radicals, which may be identical or different, chosen from halogen atoms; and the radicals: hydroxyl; free, salified or esterified carboxyl; —NH₂, —NH(alk), —N(alk)(alk); phenyl, alkyl and alkoxy, themselves optionally substituted with one or more radicals chosen from halogen atoms and hydroxyl, alkoxy, alkyl, —NH₂, —NH(alk) and —N(alk)(alk) radicals.

Even more particularly, in the products of formula (I) as defined above and below, the alkyl, alkenyl, alkynyl, aryl, heteroaryl, aralkyl or heteroaralkyl radicals can be optionally substituted with one or more radicals, which may be identical or different, chosen from halogen atoms and hydroxyl and alkoxy radicals.

A subject of the present invention is in particular the products of formula (I) above in which X represents CRa with Ra representing a hydroxyl or methoxy radical, the other substituents A1, A2, Y, R1, R2, R3 and R4 of said products of formula (I) being chosen from any one of the definitions above,

said products of formula (I) being in any of the possible racemic, enantiomeric and diastereoisomeric isomer forms, and also the addition salts with inorganic and organic acids or with inorganic and organic bases of said products of formula (I).

A subject of the present invention is in particular the products of formula (I) above in which X represents CRa with Ra representing a hydroxyl radical, the other substituents A1, A2, Y, R1, R2, R3 and R4 of said products of formula (I) being chosen from any one of the definitions above,

said products of formula (I) being in any of the possible racemic, enantiomeric and diastereoisomeric isomer forms, and also the addition salts with inorganic and organic acids or with inorganic and organic bases of said products of formula (I).

Products of formula (I) as defined in any one of the preceding claims, in which:

A1 and A2 represent CH,

X represents CRa with Ra representing a hydroxyl or methoxy radical,

Y represents CRb with Rb representing a hydrogen or bromine atom, or a —(CH₃)n-W—R5 radical with R5 chosen from alkyl, phenyl or phenylalkyl radicals optionally substituted with one or more radicals chosen from halogen atoms and alkyl radicals,

R1, R2, R3 and R4 represent a hydrogen atom,

W represents a single bond, C(O), C(O)—O, C(O)—NH; NH or —CH═N—,

n represents 0 or 1;

said products of formula (I) being in any of the possible racemic, enantiomeric and diastereoisomeric isomer forms, and also the addition salts with inorganic and organic acids or with inorganic and organic bases of said products of formula (I).

Products of formula (I) as defined in any one of the preceding claims, in which:

A1 and A2 represent CH,

X represents CRa with Ra representing a hydroxyl radical,

Y represents CRb with Rb representing a hydrogen or bromine atom, or a —W—R5 radical with R5 chosen from optionally substituted alkyl or phenylalkyl radicals,

R1, R2, R3 and R4 represent a hydrogen atom;

W having the meaning indicated above,

said products of formula (I) being in any of the possible racemic, enantiomeric and diastereoisomeric isomer forms, and also the addition salts with inorganic and organic acids or with inorganic and organic bases of said products of formula (I).

A subject of the present invention is in particular the products of formula (I) above in which Y represents CRb with Rb representing a hydrogen or bromine atom, the other substituents A1, A2, X, R1, R2, R3 and R4 of said products of formula (I) being chosen from any one of the definitions above,

said products of formula (I) being in any of the possible racemic, enantiomeric and diastereoisomeric isomer forms, and also the addition salts with inorganic and organic acids or with inorganic and organic bases of said products of formula (I).

A subject of the present invention is in particular the products of formula (I) above in which A1 and A2 represent CH,

X represents CRa with Ra representing a hydroxyl radical,

Y represents CRb with Rb representing a hydrogen or bromine atom,

R1, R2, R3, and R4 represent a hydrogen atom,

said products of formula (I) being in any of the possible racemic, enantiomeric and diastereoisomeric isomer forms, and also the addition salts with inorganic and organic acids or with inorganic and organic bases of said products of formula (I).

Among the products corresponding to formula (I) defined above, mention may be made of the products having the following names:
4-(1,2-benzisoxazol-3-yl)benzene-1,3-diol
4-(1,2-benzisoxazol-3-yl)-6-bromobenzene-1,3-diol
5-(1,2-benzisoxazol-3-yl)-2,4-dihydroxybenzenecarboxylic acid methyl ester
—N1-phenylmethyl-5-(1,2-benzisoxazol-3-yl)-2,4-dihydroxybenzene-carboxamide
1-{4-[(1,2-benzisoxazol-3-yl)-(1,3-dihydroxyphenyl]}-2-phenyl-ethanone
4-(1,2-benzisoxazol-3-yl)-6-ethylbenzene-1,3-diol
4-(1,2-benzisoxazol-3-yl)-6-(2-phenylethyl)benzene-1,3-diol
4-(1,2-benzisoxazol-3-yl)-6-(phenylamino)methylbenzene-1,3-diol
4-(1,2-benzisoxazol-3-yl)-6-(3,4-dimethylphenylamino)methylbenzene-1,3-diol
4-(1,2-benzisoxazol-3-yl)-6-(3,4-dichlorophenylamino)methylbenzene-1,3-diol
4-(1,2-benzisoxazol-3-yl)-6-(3,4-dichlorophenyl)iminomethyl-benzene-1,3-diol
   said products of formula (I) being in any of the possible racemic, enantiomeric and diastereoisomeric isomer forms, and also the addition salts with inorganic and organic acids or with inorganic and organic bases of said products of formula (I).

Among the compounds corresponding to formula (I), mention may be made of the following products:
4-(1,2-benzisoxazol-3-yl)benzene-1,3-diol
4-(1,2-benzisoxazol-3-yl)-6-bromobenzene-1,3-diol
5-(1,2-benzisoxazol-3-yl)-2,4-dihydroxybenzenecarboxylic acid methyl ester
N1-phenylmethyl-5-(1,2-benzisoxazol-3-yl)-2,4-dihydroxybenzene-carboxamide
1-{4-[(1,2-benzisoxazol-3-yl)-(1,3-dihydroxyphenyl]}-2-phenyl-ethanone
   said products of formula (I) being in any of the possible racemic, enantiomeric and diastereoisomeric isomer forms, and also the addition salts with inorganic and organic acids or with inorganic and organic bases of said products of formula (I).

Among the compounds corresponding to formula (I), mention may more particularly be made of the following products:
4-(1,2-benzisoxazol-3-yl)benzene-1,3-diol
4-(1,2-benzisoxazol-3-yl)-6-bromobenzene-1,3-diol
   said products of formula (I) being in any of the possible racemic, enantiomeric and diastereoisomeric isomer forms, and also the addition salts with inorganic and organic acids or with inorganic and organic bases of said products of formula (I).

Mention may in particular be made of the product of formula (I) as defined above, having the following name:
4-(1,2-benzisoxazol-3-yl)-6-(3,4-dimethylphenylamino)methylbenzene-1,3-diol
   said product of formula (I) being in any of the possible racemic, enantiomeric and diastereoisomeric isomer forms, and also the addition salts with inorganic and organic acids or with inorganic and organic bases of said product of formula (I).

The subject of the invention is also processes for preparing products of formula (I) as defined above.

In general, products of formula (I) in accordance with the invention can be prepared according to the various methods described by K. H. Wunsch and A. J. Boulton in Advances in Heterocyclic Chemistry Vol. 8, 277-302.

In general, products of formula (I) in accordance with the invention can be advantageously prepared by at least one of the six general methods of synthesis below.

A first general method of synthesis involves, as a key step, the formation of the 1,2-benzisoxazole ring by closure between the oxygen atom 1 and the carbon atom 7a; in particular by cyclization of ortho-halo or ortho-nitrobenzoyloximes according to General Scheme 1, using the methods described in J. Med. Chem. (1982), 25, 36.

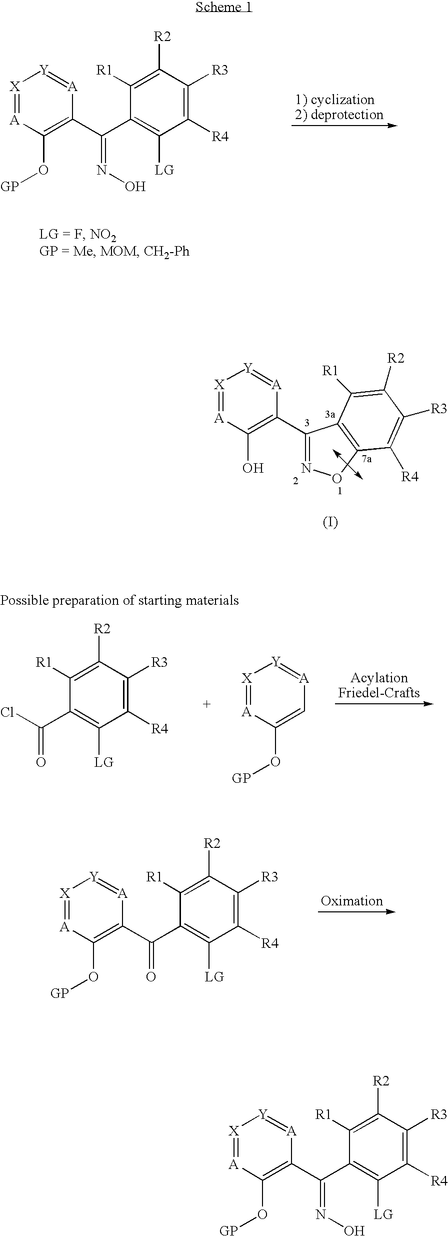

A second general method of synthesis involves, as a key step, the formation of the 1,2-benzisoxazole ring by closure between the oxygen atom in the 1-position and the nitrogen atom in the 2-position; in particular, by cyclization of activated ortho-hydroxybenzoyloxime derivatives, such as the acetates or the sulphonates, according to General Scheme 2, using in particular the methods described in J. Med. Chem. (1982), 25, 36.

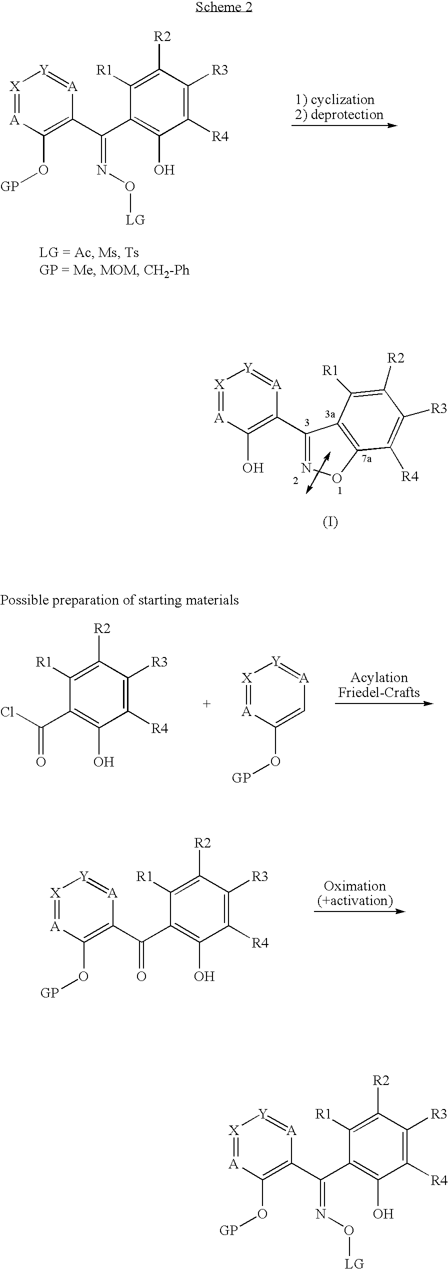

A third general method of synthesis involves, as a key step, the formation of the 1,2-benzisoxazole ring by simultaneous creation of the bonds between, firstly, the oxygen atom 1 and the carbon atom 7a and, secondly, the carbon atoms 3 and 3a; in particular, by reaction of a benzyne with a nitrile oxide according to General Scheme 3, using the methods described in Adv. Heterocycl. Chem. 1967, 8, 277 or Adv. Heterocycl. Chem. 1981, 29, 1.

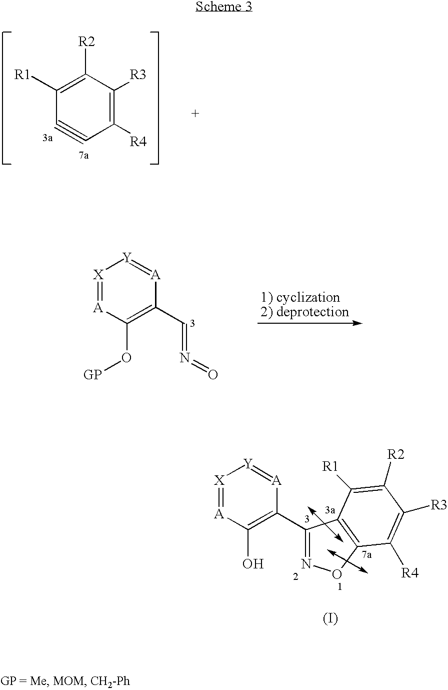

A fourth general method of synthesis involves, as a key step, the formation of the 1,2-benzisoxazole ring by closure between the nitrogen atom in the 2-position and the carbon atom in the 3-position; in particular by transoximation followed by cyclization of the 2-[(isopropylideneamino)oxy]benzophenones according to General Scheme 4; using in particular the method described in J. Org. Chem. 1984, 49, 180.

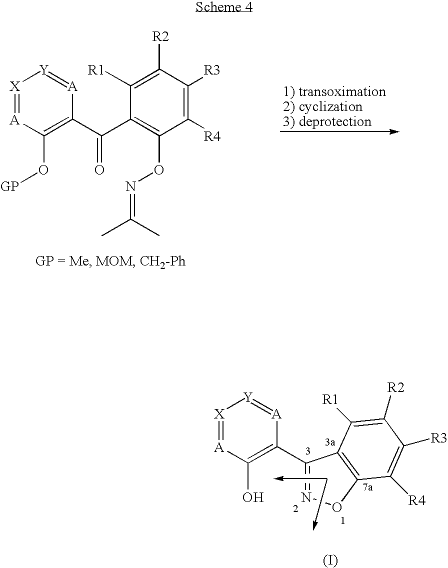

A fifth general method of synthesis involves, as a key step, a coupling reaction between a 3-halo-1,2-benzisoxazole and an “organometallic” aryl or heteroaryl derivative; in particular, according to General Scheme 5, a Suzuki-type coupling reaction, catalysed by a palladium(0) complex, between a 3-bromo-1,2-benzisoxazole and a suitably selected arylboronic acid, carrying out the procedure, for example, according to Synth. Commun. 1981, 11, 513.

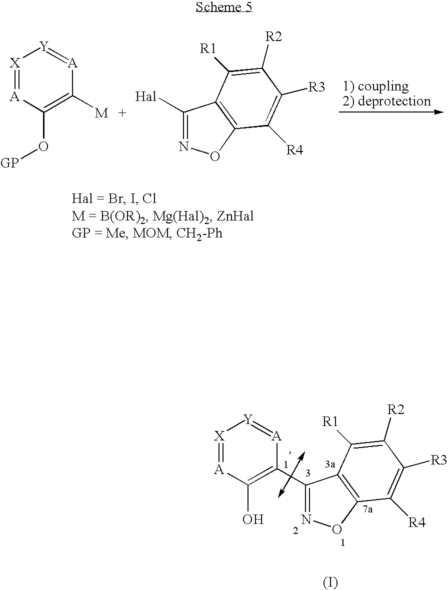

The organometallic derivatives of the aryl- or heteroarylaromatic compounds are either commercial, or prepared as described in the literature, or prepared according to the general methods known to those skilled in the art. The 3-halo-1,2-benzisoxazole compounds are either commercial, or prepared as described in the literature, or prepared according to the general methods known to those skilled in the art.

A sixth general method of synthesis, that is particularly advantageous in the context of the invention, consists in substituting either the aryl ring or the 1,2-benzisoxazole ring after the formation of a suitably selected 3-aryl-1,2-benzisoxazole compound, or in modifying the nature of these substituents either of the aryl ring or of the 1,2-benzisoxazole ring according to the general methods known to those skilled in the art, in particular those described in:
Comprehensive Heterocyclic Chemistry, by A. Katritsky et al. (Pergamon Press)
Comprehensive Heterocyclic Chemistry, by D. Barton et al. (Pergamon Press);
Advanced Organic Chemistry, by J. Marsh (Wiley Interscience)

By way of example, it is particularly advantageous, in order to prepare the preferred compounds of the invention in which X=C—OH and A=CH, to carry out the procedure according to General Scheme 6:

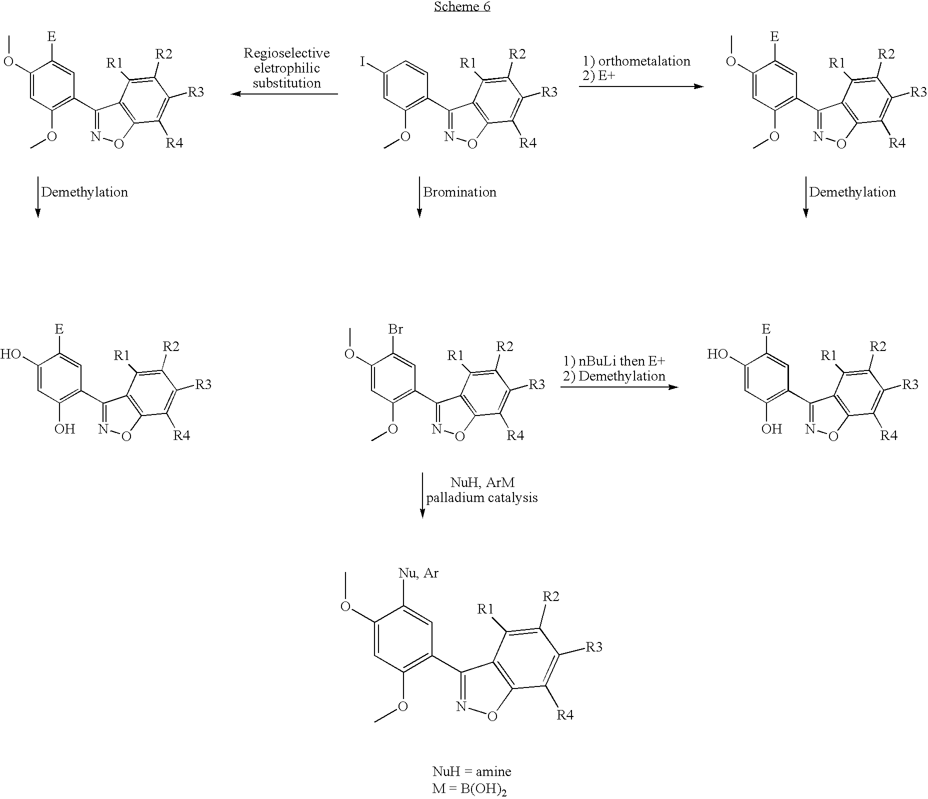

This method is particularly appropriate in the context of the present invention in the following cases:
the radicals R₁, R₂, R₃ and R₄ all represent a hydrogen atom;
none of the radicals R₁, R₂, R₃ and R₄ represents a group capable of activating the benzo ring of the benzisoxazole with respect to reactions of aromatic electrophilic substitution type according to Friedel-Crafts, such as, in a nonlimiting respect, alkyl or alkoxy radicals;
none of the radicals R₁, R₂, R₃ and R₄ represents a group capable of promoting an orthometalation reaction followed by electrophilic trapping on the benzo ring of the benzisoxazole, such as, in a nonlimiting respect, an alkoxy radical, a trifluoroacetamido radical or a tert-butoxycarbonylamino radical;
none of the radicals R₁, R₂, R₃ and R₄ represents a halogen atom capable of being exchanged with a metal.

The products that are the subject of the present invention have advantageous pharmacological properties: it has been noted that they in particular possess protein-inhibiting properties. Among these proteins, mention is in particular made of Hsp90.

A subject of the invention is therefore the application, as medicinal products, of the pharmaceutically acceptable products of general formula (I).

A subject of the invention is in particular the application, as medicinal products, of the products having the following names:
4-(1,2-benzisoxazol-3-yl)benzene-1,3-diol
4-(1,2-benzisoxazol-3-yl)-6-bromobenzene-1,3-diol
5-(1,2-benzisoxazol-3-yl)-2,4-dihydroxybenzenecarboxylic acid methyl ester
N1-phenylmethyl-5-(1,2-benzisoxazol-3-yl)-2,4-dihydroxybenzene-carboxamide
1-{4-[(1,2-benzisoxazol-3-yl)-(1,3-dihydroxyphenyl]}-2-phenyl-ethanone
4-(1,2-benzisoxazol-3-yl)-6-ethylbenzene-1,3-diol
4-(1,2-benzisoxazol-3-yl)-6-(2-phenylethyl)benzene-1,3-diol
4-(1,2-benzisoxazol-3-yl)-6-(phenylamino)methylbenzene-1,3-diol 04-(1,2-benzisoxazol-3-yl)-6-(3,4-dimethylphenylamino)methylbenzene-1,3-diol
4-(1,2-benzisoxazol-3-yl)-6-(3,4-dichlorophenylamino)methylbenzene-1,3-diol
4-(1,2-benzisoxazol-3-yl)-6-(3,4-dichlorophenyl)iminomethylbenzene-1,3-diol
   and also the prodrugs thereof, said products of formula (I) being in any of the possible racemic, enantiomeric and diastereoisomeric isomer forms, and also the pharmaceutically acceptable addition salts with inorganic and organic acids or with inorganic and organic bases of said products of formula (I).

A subject of the invention is in particular the application, as medicinal products, of the products having the following names:
4-(1,2-benzisoxazol-3-yl)benzene-1,3-diol
4-(1,2-benzisoxazol-3-yl)-6-bromobenzene-1,3-diol
5-(1,2-benzisoxazol-3-yl)-2,4-dihydroxybenzenecarboxylic acid methyl ester
N1-phenylmethyl-5-(1,2-benzisoxazol-3-yl)-2,4-dihydroxybenzenecarboxamide
1-{4-[(1,2-benzisoxazol-3-yl)-(1,3-dihydroxyphenyl]}-2-phenylethanone
   and also the prodrugs thereof, said products of formula (I) being in any of the possible racemic, enantiomeric and diastereoisomeric isomer forms, and also the pharmaceutically acceptable addition salts with inorganic and organic acids or with inorganic and organic bases of said products of formula (I).

A subject of the invention is more particularly the application, as medicinal products, of the products having the following names:
4-(1,2-benzisoxazol-3-yl)benzene-1,3-diol
4-(1,2-benzisoxazol-3-yl)-6-bromobenzene-1,3-diol
   and also the prodrugs thereof, said products of formula (I) being in any of the possible racemic, enantiomeric and diastereoisomeric isomer forms, and also the pharmaceutically acceptable addition salts with inorganic and organic acids or with inorganic and organic bases of said products of formula (I).

A subject of the invention is in particular the application, as a medicinal product, of the product 4-(1,2-benzisoxazol-3-yl)benzene-1,3-diol as defined above, and also the prodrugs thereof, said product of formula (I) being in any of the possible racemic, enantiomeric and diastereoisomeric isomer forms, and also the pharmaceutically acceptable addition salts with inorganic and organic acids or with inorganic and organic bases of said product of formula (I).

The products can be administered parenterally, buccally, perlingually, rectally or topically.

A subject of the invention is also the pharmaceutical compositions, characterized in that they contain, as active ingredient, at least one of the medicinal products of formula (I).

A subject of the invention is thus the pharmaceutical compositions as defined above, characterized in that they are used as medicinal products, in particular for cancer chemotherapy.

A subject of the invention is thus the pharmaceutical compositions as defined above, also containing active ingredients of other medicinal products for cancer chemotherapy.

These compositions can be in the form of injectable solutions or suspensions, of tablets, of coated tablets, of capsules, of syrups, of suppositories, of creams, of ointments and of lotions. These pharmaceutical forms are prepared according to the usual methods. The active ingredient can be incorporated into excipients normally used in these compositions, such as aqueous or nonaqueous carriers, talc, gum arabic, lactose, starch, magnesium stearate, cocoa butter, fatty substances of animal or plant origin, paraffin derivatives, glycols, the various wetting agents, dispersants or emulsifiers, or preserving agents.

The usual dose, which can be varied according to the individual treated and the condition in question, may be, for example, from 10 mg to 500 mg per day in humans, orally.

The products corresponding to general formula (I) as defined above thus exhibit a substantial Hsp90 chaperone-inhibiting activity.

A subject of the present invention is thus also the use of products of formula (I) as defined above or of pharmaceutically acceptable salts of said products of formula (I), for preparing a medicinal product for use in preventing or treating a disease characterized by a disturbance in Hsp90 protein activity.

A subject of the present invention is thus the use of products of formula (I) as defined above or of pharmaceutically acceptable salts of said products of formula (I), for preparing medicinal products for use in inhibiting Hsp90 protein activity.

A subject of the present invention is thus the use of products of formula (I) as defined above or of pharmaceutically acceptable salts of said products of formula (I), in which the disease to be prevented or treated is in a mammal.

Tests given in the experimental section hereinafter illustrate the inhibitory activity of products of the present invention with respect to such proteins.

A subject of the present invention is thus also the use of products of formula (I) as defined above or of pharmaceutically acceptable salts of said products of formula (I), for preparing a medicinal product for use in treating cancers.

A subject of the present invention is thus also the use of products of formula (I) as defined above or of pharmaceutically acceptable salts of said products of formula (I), in which the disease to be treated is a solid or liquid tumour cancer.

The properties of the products of formula (I) of the present invention therefore make them usable as medicinal products in particular for the treatment of malignant tumours.

Among these cancers, the present invention is interested most particularly in the treatment of solid tumours and in the treatment of cancers resistant to cytotoxic agents.

A subject of the present invention is thus also the use of products of formula (I) as defined above or of pharmaceutically acceptable salts of said products of formula (I), in which the disease to be treated is a cancer resistant to cytotoxic agents.

A subject of the present invention is thus also the use of products of formula (I) as defined above or of pharmaceutically acceptable salts of said products of formula (I), for preparing a medicinal product for use in treating cancers among which are lung cancer, breast cancer and ovarian cancer, glioblastomas, chronic myeloid leukaemias, acute lymphoblastic leukaemias, prostate cancer, pancreatic cancer and colon cancer, metastatic melanomas, thyroid tumours and renal carcinomas.

Thus, among the main potential indications for Hsp90 inhibitors, mention may, in a nonlimiting capacity, be made of:
“non small cell” lung cancers, breast cancers, ovarian cancers and glioblastomas that overexpress EGF-R or HER2;
chronic myeloid leukaemias that overexpress Bcr-Abl;
acute lymphoblastic leukaemias that overexpress Flt-3;
breast cancers, prostate cancers, lung cancers, pancreatic cancers, colon cancers or ovarian cancers that overexpress Akt;
metastatic melanomas and thyroid tumours that overexpress the mutated form of the B-Raf protein;
androgen-dependent and androgen-independent prostate cancers;
oestrogen-dependent and oestrogen-independent breast cancers;
renal carcinomas that overexpress HIF-1a or the mutated c-met protein, etc.

The present invention also relates to the use of products of formula (I) as defined above or of pharmaceutically acceptable salts of said products of formula (I), for preparing a medicinal product for use in cancer chemotherapy.

As medicinal products according to the present invention for use in cancer chemotherapy, the products of formula (I) according to the present invention can be used alone or in combination with chemotherapy or radiotherapy or alternatively in combination with other therapeutic agents.

A subject of the present invention is thus also the use of products of formula (I) as defined above or of pharmaceutically acceptable salts of said products of formula (I), for preparing medicinal products for use in cancer chemotherapy, used alone or in combination.

The present invention thus relates in particular to the pharmaceutical compositions as defined above, also containing active ingredients of other medicinal products for cancer chemotherapy.

Such therapeutic agents may be commonly used antitumour agents.

A subject of the present invention is thus also the use of products of formula (I) as defined above or of pharmaceutically acceptable salts of said products of formula (I), for preparing medicinal products intended to be used alone or in combination with chemotherapy or radiotherapy or alternatively in combination with other therapeutic agents.

A subject of the present invention is thus also the use of products of formula (I) as defined above or of pharmaceutically acceptable salts of said products of formula (I), in which the therapeutic agents may be commonly used antitumour agents.

A subject of the present invention is in particular the use of the product 4-(1,2-benzisoxazol-3-yl)benzene-1,3-diol as defined above, or of pharmaceutically acceptable salts of this product, according to any one of the uses defined above.

As examples of known inhibitors of protein kinases, mention may in particular be made of butyrolactone, flavopiridol, 2-(2-hydroxyethylamino)-6-benzyl-amino-9-methylpurine, olomucine, Glivec and Iressa.

The products of formula (I) according to the present invention can thus also be advantageously used in combination with antiproliferative agents: by way of examples of such antiproliferative agents, but without, however being limited to this list, mention may be made of aromatase inhibitors, antioestrogens, topoisomerase I inhibitors, topoisomerase II inhibitors, agents that are active on microtubules, alkylating agents, histone deacetylase inhibitors, farnesyl transferase inhibitors, COX-2 inhibitors, MMP inhibitors, mTOR inhibitors, antineoplastic antimetabolites, platinum compounds, compounds that decrease protein kinase activity and also anti-angiogenic compounds, gonadorelin agonists, anti-androgens, bengamides, biphosphonates and trastuzumab.

By way of examples, mention may thus be made of anti-microtubule agents such as taxoids, vinca-alkaloids, alkylating agents such as cyclophosphamide, DNA-intercalating agents such as cis-platinum, agents that interact on topoisomerase, such as camptothecin and derivatives, anthracyclines such as adriamycin, and antimetabolites such as 5-fluorouracil and derivatives and analogues.

The present invention therefore relates to products of formula (I) as protein kinase inhibitors, said products of formula (I) being in any of the possible racemic, enantiomeric and diastereoisomeric isomer forms, and also the pharmaceutically acceptable addition salts with inorganic and organic acids or with inorganic and organic bases of said products of formula (I) and also the prodrugs thereof.

The present invention relates in particular to products of formula (I) as defined above, as Hsp90 inhibitors, said products of formula (I) being in any of the possible racemic, enantiomeric and diastereoisomeric isomer forms, and also the pharmaceutically acceptable addition salts with inorganic and organic acids or with inorganic and organic bases of said products of formula (I) and also the prodrugs thereof.

The present invention relates in particular to the product 4-(1,2-benzisoxazol-3-yl)benzene-1,3-diol as an Hsp90 inhibitor, said product being in any of the possible racemic, enantiomeric and diastereoisomeric isomer forms, and also the pharmaceutically acceptable addition salts with inorganic and organic acids or with inorganic and organic bases of said products of formula (I) and also the produgs thereof.

The products of formula (I) according to the present invention can be prepared by the application or the adaptation of known methods, and in particular of the methods described in the literature, for instance those described by R. C. Larock in: Comprehensive Organic Transformations, VCH publishers, 1989.

In the reactions described hereinafter, it may be necessary to protect reactive functional groups such as, for example, hydroxyl, amino, imino, thio or carboxyl groups, when the latter are desired in the final product but when their participation is not desired in the reactions for synthesizing the products of formula (I). Conventional protective groups can be used in accordance with the usual standard practices, such as those described, for example, by T. W. Greene and P. G. M. Wuts in “Protective Groups in Organic Chemistry” John Wiley and Sons, 1991.

The examples for which the preparation is given below illustrate the present invention without however limiting it.

### EXAMPLES ILLUSTRATING THE INVENTION

### Example 1

### 4-(1,2-Benzisoxazol-3-yl)benzene-1,3-diol

400 mg of 2-(1,2-benzisoxazol-3-yl)-5-methoxyphenol are dissolved in 15 ml of dichloromethane and then 4.97 ml of a molar solution of boron tribromide in dichloromethane are added, dropwise, at ambient temperature. After stirring at ambient temperature for 2 hours, 1.66 ml of the 1M boron tribromide solution are again added, dropwise, and the mixture is brought to reflux for 2 hours. After cooling, 50 ml of water are poured in, the dichloromethane is concentrated, and then the residue is stirred in water. The precipitate thus formed is filter-dried, washed successively with water, then with petroleum ether, and dried under a hood in the open air. 270 mg of 4-(1,2-benzisoxazol-3-yl)benzene-1,3-diol are thus obtained in the form of an amorphous white solid, the characteristics of which are as follows:

melting point (Kofler bench)=124° C.

1H NMR spectrum (300 MHz)-δ in ppm-in DMSO-d6:

6.42 (dd, J=2.5 and 8.5 Hz, 1H); 6.54 (d, J=2.5 Hz, 1H); 7.39 (m, 2H); 7.65 (dt, J=1.0 and 8.0 Hz, 1H); 7.75 (broad d, J=8.0 Hz, 1H); 7.87 (broad d, J=8.0 Hz, 1H); from 9.55 to 10.1 (very broad m, 2H).

The 2-(1,2-benzisoxazol-3-yl)-5-methoxyphenol can be prepared from 2,2′-dihydroxy-4-methoxybenzophenone by carrying out the process under the conditions described in J. Org. Chem. 1983, 48, 2613-15.

### Example 2

### 4-(1,2-Benzisoxazol-3-yl)-6-bromobenzene-1,3-diol

Step 1: 2.163 g of acetone oxime are dissolved in 75 ml of DMF, in a 250 ml three-necked flask, and 3.317 g of potassium tert-butanolate are added. 7 g of (2,4-dimethoxyphenyl)-(2-fluorophenyl)methanone are then added slowly in approximately 2 h and the mixture is then stirred at ambient temperature for 3 days. After concentration under reduced pressure, the reaction residue is taken up with 100 ml of diethyl ether, washed 3 times with 50 ml of water, dried over magnesium sulphate and concentrated to dryness under reduced pressure. The orangey-brown oil thus obtained is purified by flash chromatography on silica gel, elution being carried out with a mixture of ethyl acetate and cyclohexane (20/80 by volume). 7.85 g of (2,4-dimethoxyphenyl)-{2-[(isopropylidene)amino]oxyphenyl}methanone are thus obtained in the form of an orange oil, which is used as it is in the subsequent step.

The (2,4-dimethoxyphenyl)-(2-fluorophenyl)methanone can be prepared by carrying out the process according to WO 9420869.

Step 2: 7.834 g of (2,4-dimethoxyphenyl)-{2-[(isopropylidene)amino]oxyphenyl}methanone are dissolved in 150 ml of acetonitrile, in a 250 ml three-necked flask, under an argon atmosphere, and the mixture is then cooled to around 5° C. and 127 ml of a 1.4M aqueous hydrochloric acid solution are slowly added. The mixture is then heated at 80° C. for 3 hours. After concentration under reduced pressure, the reaction medium is taken with 100 ml of ethyl acetate and 20 ml of water. The organic phase is separated by settling out, washed with water and then with a saturated ammonium chloride solution, dried over sodium sulphate and concentrated to dryness under reduced pressure. After purification by flash chromatography on silica gel, elution being carried out with a mixture of cyclohexane and ethyl acetate (90/10 by volume), 4.5 g of 3-(2,4-dimethoxyphenyl)-1,2-benzisoxazole are obtained in the form of an off-white amorphous solid, the characteristics of which are as follows:

Mass spectrum (EI): m/z=255 (M+)

Step 3: 1 g of 3-(2,4-dimethoxyphenyl)-1,2-benzisoxazole is dissolved in 40 ml of chloroform, in a 100 ml three-necked flask, under an argon atmosphere. After cooling to 0° C., a solution of 0.69 g of bromine in 5 ml of chloroform is added, dropwise, and stirring is carried out for 1 h at 0° C. After neutralization with a saturated aqueous sodium hydrogen carbonate solution, the organic phase is separated by settling out, washed with water, dried over magnesium sulphate and concentrated to dryness under reduced pressure. 1.3 g of 3-(5-bromo-2,4-dimethoxyphenyl)-1,2-benzisoxazole are thus obtained in the form of a beige powder, the characteristics of which are as follows:

Mass spectrum (EI): m/z=334 (M+)

Step 4: The process is carried out as in Example 1, but using 334 mg of 3-(5-bromo-2,4-dimethoxyphenyl)-1,2-benzisoxazole and 5 ml of a molar solution of boron tribromide in 20 ml of dichloromethane. By purifying by flash chromatography on silica gel, elution being carried out with a mixture of cyclohexane and ethyl acetate (90/10 by volume), and by recovering the second fraction eluted, 70 mg of 4-(1,2-benzisoxazol-3-yl)-6-bromobenzene-1,3-diol are obtained in the form of an off-white solid, the characteristics of which are as follows:

Mass spectrum (EI): m/z=306 (M+)

### Example 3

### 5-(1,2-Benzisoxazol-3-yl)-2,4-dihydroxybenzenecarboxylic acid methyl ester

Step 1: 334 mg of 3-(6-bromo-2,4-dimethoxyphenyl)-1,2-benzisoxazole, obtained in Step 3 of Example 1, are dissolved in 10 ml of anhydrous THF, in a 50 ml three-necked flask, under an argon atmosphere, and the solution is then cooled to −70° and 0.69 ml of a 1.6M solution of n-butyllithium in hexane is added dropwise. The mixture is stirred at −70° C. for 30 minutes, then 0.234 ml of methyl chloroformate is run in slowly and, finally, the mixture is stirred for 2 hours while allowing a return to ambient temperature. The mixture is subsequently brought to pH=6 by the addition of a saturated aqueous ammonium chloride solution and 20 ml of ethyl acetate are added. The organic phase is separated by settling out, washed with water, dried over magnesium sulphate and concentrated to dryness under reduced pressure. After purification by flash chromatography on silica gel, elution being carried out with a mixture of cyclohexane and ethyl acetate (80/20 by volume), 180 mg of 5-(1,2-benzisoxazol-3-yl)-2,4-dimethoxybenzenecarboxylic acid methyl ester are obtained in the form of an amorphous beige solid, the characteristics of which are as follows:

Mass spectrum (EI): m/z=313 (M+)

Step 2: The process is carried out as in Example 1, but using 150 mg of 5-(1,2-benzisoxazol-3-yl)-2,4-dimethoxybenzenecarboxylic acid methyl ester and 1.198 ml of a molar solution of boron tribromide in 5 ml of dichloromethane. By purifying by flash chromatography on silica gel, elution being carried out with a mixture of cyclohexane and ethyl acetate (90/10 by volume), and by recovering the second fraction eluted, which is crystallized from 2 ml of diisopropyl ether, 75 mg of 5-(1,2-benzisoxazol-3-yl)-2,4-dihydroxybenzenecarboxylic acid methyl ester are obtained in the form of an off-white solid, the characteristics of which are as follows:

Mass spectrum (EI): m/z=285 (M+)

### Example 4

### N1-Phenylmethyl-5-(1,2-benzisoxazol-3-yl)-2,4-dihydroxybenzenecarboxamide

Step 1: 260 mg of 5-(1,2-benzisoxazol-3-yl)-2,4-dimethoxybenzenecarboxylic acid methyl ester, obtained in Step 1 of Example 3, are dissolved in 4 ml of methanol and 1.5 ml of water, in a 25 ml three-necked flask, under an argon atmosphere, then 41.7 mg of lithium hydroxide are added and the mixture is stirred at ambient temperature overnight. After concentration under reduced pressure, the reaction medium is taken up in 25 ml of water and washed with 10 ml of diethyl ether. The aqueous phase is acidified to pH=1 by the addition of a 1N aqueous hydrochloric acid solution. The precipitate formed is filter-dried, washed with water and dried in the oven at 50° C. 210 mg of 5-(1,2-benzisoxazol-3-yl)-2,4-dimethoxybenzenecarboxylic acid are thus obtained in the form of an ecru solid, the characteristics of which are as follows:

Mass spectrum (EI): m/z=299 (M+)

Step 2: A solution of 175 mg of 5-(1,2-benzisoxazol-3-yl)-2,4-dimethoxybenzenecarboxylic acid and 136 mg of N-benzylamine in 10 ml of dichloromethane are stirred overnight at ambient temperature, in the presence of 123 mg of EDCI and 87 mg of HOBT, in a 25 ml three-necked flask. After the addition of 25 ml of water and of 25 ml of dichloromethane, the organic phase is separated by settling out, washed with water, dried over magnesium sulphate and concentrated under reduced pressure. By purifying by flash chromatography on silica gel, elution being carried out with a mixture of cyclohexane and ethyl acetate (90/10 by volume), 220 mg of N1-phenylmethyl-5-(1,2-benzisoxazol-3-yl)-2,4-dimethoxybenzenecarbox-amide are obtained in the form of a beige solid, the characteristics of which are as follows:

Mass spectrum (EI): m/z=388 (M+)

Step 3: The process is carried out as in Example 1, but using 185 mg of N1-phenylmethyl-5-(1,2-benzisoxazol-3-yl)-2,4-dimethoxybenzenecarbox-amide and 1.19 ml of a molar solution of boron tribromide in 5 ml of dichloromethane. By purifying by flash chromatography on silica gel, elution being carried out with a mixture of cyclohexane and ethyl acetate (90/10 by volume), and by recovering the first fraction eluted, which is crystallized from 2 ml of methanol, 25 mg of N1-phenylmethyl-5-(1,2-benzisoxazol-3-yl)-2,4-dihydroxybenzenecarboxamide methyl ester are obtained in the form of a light beige solid, the characteristics of which are as follows:

1H NMR spectrum at 400 MHz-δ in ppm-DMSO-D6: 4.53 (broad s, 2H); 6.70 (s, 1H); 7.21 (m, 2H); 7.29 (m, 3H); 7.31 (partially masked m, 1H); 7.46 (t, J=8.0 Hz, 1H); 7.71 (t, J=8.0 Hz, 1H); 7.43 (d, J=8.0 Hz, 1H); 7.44 (d, J=8.0 Hz, 1H); 8.50 (s, 1H); 11.45 (broad s, 1H); 11.95 (broad s, 1H).

Mass spectrum (EI): m/z=360 (M+)

### Example 5

### 1-{4-[(1,2-Benzisoxazol-3-yl)-(1,3-dihydroxyphenyl]}-2-phenylethanone

511 mg of 3-(2,4-dimethoxyphenyl)-1,2-benzisoxazole, obtained in Step 2 of Example 2, are dissolved in 20 ml of 1,2-dichloroethane, in a 100 ml three-necked flask, under an argon atmosphere, and then 309 mg of phenylacetic acid chloride and 404 mg of aluminium chloride are added successively. The reaction medium is then heated at 80° C. for 8 hours. After cooling, the reaction medium is run into 100 ml of water and 10 ml of 5N hydrochloric acid and then stirred for 10 minutes. The organic phase is separated by settling out and then extraction is carried out three times with 50 ml of dichloromethane. The combined organic phases are washed with water, dried over magnesium sulphate and concentrated under reduced pressure. The reaction medium is purified on silica gel, elution being carried out with a mixture of cyclohexane and dichloromethane (6/1 by volume). The second fraction eluted is recovered, and is subsequently crystallized from 3 ml of diisopropyl ether. 60 mg of 1-{4-[(1,2-benzisoxazol-3-yl)-(1,3-dihydroxy-phenyl]}-2-phenylethanone are thus obtained in the form of a light grey solid, the characteristics of which are as follows:

1H NMR spectrum at 400 MHz-δ in ppm-DMSO-D6: 4.88 (s, 2H); 6.55 (s, 1H); from 7.18 to 7.35 (m, 5H); 7.39 (t, J=7.5 Hz, 1H); 7.66 (t, J=7.5 Hz, 1H); 7.75 (d, J=8.0 Hz, 1H); 7.78 (d, J=8.0 Hz, 1H); 8.22 (s, 1H); from 11.6 to 12.8 (very broad m, 2H)

Mass spectrum (EI): m/z=345 (M+)

### Example 6

### 4-(1,2-Benzisoxazol-3-yl)-6-ethylbenzene-1,3-diol

Step 1: 1.03 g of 3-(5-bromo-2,4-dimethoxyphenyl)-1,2-benzisoxazole are dissolved in 35 ml of THF, in a 100 ml three-necked flask, under an argon atmosphere, and then the solution is cooled to −78° C. 2.063 ml of a 1.6M solution of n-butyllithium in hexane are then added slowly at −78° C., and then, after stirring for 1 hour at −78° C., a solution of 1.404 g of iodoethane in 5 ml of THF is added, still at −78° C. The mixture is allowed to return to ambient temperature and stirring is carried out for 20 hours. The reaction medium is then run into 100 ml of a saturated aqueous ammonium chloride solution and is extracted 3 times with 25 ml of ethyl acetate. The combined organic phases are washed with water, dried over magnesium sulphate and concentrated under reduced pressure. After purification by flash chromatography on silica gel, elution being carried out with a mixture of cyclohexane and diisopropyl ether (90/10 by volume), 700 mg of an orangey-coloured oil, containing mainly, by NMR, 3-(5-ethyl-2,4-dimethoxyphenyl)-1,2-benzisoxazole, are obtained, which oil is used as it is in the subsequent step.

Step 2: The process is carried out as in Example 1, but using 1 g of 3-(5-ethyl-2,4-dimethoxyphenyl)-1,2-benzisoxazole and 8.83 ml of a molar solution of boron tribromide in 40 ml of dichloromethane. By purifying by flash chromatography on silica gel, elution being carried out with a mixture of cyclohexane and diisopropyl ether (90/10 by volume), and by recovering the third fraction eluted and then crystallizing it from pentane, 270 mg of 4-(1,2-benzisoxazol-3-yl)-6-ethylbenzene-1,3-diol are obtained in the form of an off-white solid, the characteristics of which are as follows:

1H NMR spectrum at 400 MHz-δ in ppm-DMSO-D6: 1.13 (t, J=7.0 Hz, 3H); from 2.44 to 2.54 (masked m, 2H); 6.58 (s, 1H); 7.23 (s, 1H); 7.36 (t, J=8.0 Hz, 1H); 7.62 (t, J=8.0 Hz, 1H); 7.73 (d, J=8.0 Hz, 1H); 7.87 (d, J=8.0 Hz, 1H); 9.71 (broad s, 2H)

Mass spectrum (EI): m/z=255 (M+)

### Example 7

### 4-(1,2-Benzisoxazol-3-yl)-6-(2-phenylethyl)benzene-1,3-diol

Step 1: 1.51 g of 3-(5-bromo-2,4-dimethoxyphenyl)-1,2-benzisoxazole are dissolved in 75 ml of DMF, in a 100 ml three-necked flask, under an argon atmosphere, and then 1.10 g of (2E-phenylethenyl)boronic acid, 578 mg of tetrakis(triphenylphosphine)palladium and 1.26 g of sodium hydrogen carbonate are added successively. The reaction medium is brought to 80° C. for 20 hours. After concentration under reduced pressure, the residue is taken up with 50 ml of water and 50 ml of ethyl acetate. After filtration of an insoluble material, which is washed twice with 20 ml of ethyl acetate, the filtrate is separated by settling out; the organic phase is then washed with a saturated aqueous sodium chloride solution, dried over magnesium sulphate and concentrated under reduced pressure. After purification by flash chromatography on silica gel, elution being carried out with a mixture of cyclohexane and diisopropyl ether, 700 mg of 3-[5-(2E-phenylethenyl)-2,4-dimethoxyphenyl]-1,2-benzisoxazole are obtained in the form of a yellow oil, which is used as it is in the subsequent step, and the characteristics of which are as follows:

Mass spectrum (EI): m/z=357 (M+)

Step 2: A solution of 700 mg of 3-[5-(2E-phenylethenyl)-2,4-dimethoxyphenyl]-1,2-benzisoxazole, obtained in the preceding step, in 44 ml of ethanol is stirred for 6 hours, under a hydrogen pressure of 1 bar, in a 100 ml Top45 autoclave, in the presence of 20 mg of 5% palladium-on-charcoal. After filtration of the catalyst, the solvent is concentrated under reduced pressure and the residue is purified by flash chromatography on silica gel, elution being carried out with a mixture of cyclohexane and diisopropyl ether. 500 mg of a yellow oil, which is used as it is in the subsequent step, containing mainly, by NMR, 3-[5-(2-phenyl-ethyl)-2,4-dimethoxyphenyl]-1,2-benzisoxazole, are obtained in a form the characteristics of which are as follows.

Step 3: The process is carried out as in Example 1, but using 500 mg of 3-[5-(2-phenylethyl)-2,4-dimethoxyphenyl]-1,2-benzisoxazole and 3.48 ml of a molar solution of boron tribromide in 15 ml of dichloromethane. By purifying by flash chromatography on silica gel, elution being carried out with a mixture of cyclohexane and diisopropyl ether (90/10 by volume), and by recovering the second fraction eluted and then crystallizing it from diisopropyl ether, 25 mg of 4-(1,2-benzisoxazol-3-yl)-6-(2-phenylethyl)benzene-1,3-diol are obtained in the form of a white solid, the characteristics of which are as follows:

1H RMN spectrum at 400 MHz-δ in ppm-DMSO-D6: from 2.73 to 2.79 (m, 4H); 6.60 (s, 1H); 7.17 (t, J=7.5 Hz, 1H); from 7.21 to 7.30 (m, 4H); 7.22 (s, 1H); 7.35 (t, J=8.0 Hz, 1H); 7.63 (t, J=8.0 Hz, 1H); 7.72 (d, J=8.0 Hz, 1H); 7.75 (d, J=8.0 Hz, 1H); from 7.6 to 7.9 (broad m, 2H).

Mass spectrum (EI): m/z=331 (M+)

### Example 8

### 4-(1,2-Benzisoxazol-3-yl)-6-(phenylamino)methylbenzene-1,3-diol

Step 1: 1.67 g of 3-(5-bromo-2,4-dimethoxyphenyl)-1,2-benzisoxazole are dissolved in 66 ml of THF, in a 100 ml three-necked flask, under an argon atmosphere, and then the solution is cooled to −70° C. 3.44 ml of a 1.6M solution of n-butyllithium in hexane are then added slowly at −70° C.; after stirring at −70° C. for 1 hour, 3.65 g of DMF are then added, still at −70° C. The mixture is allowed to return to ambient temperature and stirring is carried out for 20 hours. The reaction medium is then run into 100 ml of a saturated aqueous ammonium chloride solution and extracted three times with 50 ml of ethyl acetate. The combined organic phases are washed with water, dried over magnesium sulphate and concentrated under reduced pressure. After purification by flash chromatography on silica gel, elution being carried out with a mixture of cyclohexane and ethyl acetate (60/40 by volume), 0.8 g of 5-(1,2-benzoxazol-3-yl)-2,4-dimethoxybenzaldehyde is obtained in the form of a light beige solid, the characteristics of which are as follows:

Mass spectrum (EI): m/z=283 (M+)

Step 2: 100 mg of 5-(1,2-benzoxazol-3-yl)-2,4-dimethoxybenzaldehyde, obtained in the preceding step, are dissolved in 2 ml of methanol, in a 25 ml three-necked flask, under an argon atmosphere, and then 35.4 μl of aniline, 106 mg of acetic acid and 100 mg of molecular sieve 3A are added. The mixture is stirred at ambient temperature for 15 minutes; 48.8 mg of sodium cyanoborohydride are then added and the mixture is stirred for a further 20 hours at ambient temperature. After the addition of 5 ml of a saturated aqueous sodium hydrogen carbonate solution, the mixture is filtered over Celite and then extraction is carried out 3 times with 25 ml of dichloromethane. The combined organic phases are washed with water and then with a saturated aqueous sodium chloride solution, dried over magnesium sulphate and concentrated under reduced pressure. After purification by flash chromatography on silica gel, elution being carried out with a mixture of cyclohexane and ethyl acetate (60/40 by volume), 100 mg of [5-(1,2-benzisoxazol-3-yl)-2,4-dimethoxyphenyl]methylphenylamine are obtained in the form of an orangey-coloured oil, the characteristics of which are as follows:

Mass spectrum (EI): m/z=360 (M+)

Step 3: The process is carried out as in Example 1, but using 200 mg of [5-(1,2-benzisoxazol-3-yl)-2,4-dimethoxyphenyl]methylphenylamine and 1.39 ml of a molar solution of boron tribromide in 6 ml of dichloromethane. By purifying by flash chromatography on silica gel, elution being carried out with a mixture of cyclohexane and ethyl acetate (80/20 by volume), and by recovering the second fraction eluted and then crystallizing it from diisopropyl ether, 11 mg of 4-(1,2-benzisoxazol-3-yl)-6-(phenylamino)methylbenzene-1,3-diol are obtained in the form of a yellow solid, the characteristics of which are as follows:

1H NMR spectrum at 400 MHz-δ in ppm-DMSO-D6: 4.16 (d, J=5.5 Hz, 2H); 5.96 (broad t, J=5.5 Hz, 1H); 6.50 (broad t, J=7.5 Hz, 1H); 6.57 (d, J=8.5 Hz, 2H); 6.62 (s, 1H); 7.04 (dd, J=7.5 and 8.5 Hz, 2H); 7.29 (broad t, J=8.0 Hz, 1H); 7.39 (s, 1H); 7.61 (dt, J=1.0 and 8.0 Hz, 1H); 7.68 (d, J=8.0 Hz, 1H); 7.70 (d, J=8.0 Hz, 1H); from 9.65 to 10.2 (broad m, 2H).

Mass spectrum (EI): m/z=332 (M+)

### Examples 9 and 10

### 4-(1,2-Benzisoxazol-3-yl)-6-(3,4-dimethylphenylamino)methylbenzene-1,3-diol and 2-(1,2-benzisoxazol-3-yl)-4-(3,4-dimethylphenylamino)methyl-5-methoxyphenol

Step 1: The process is carried out as in Step 2 of Example 8, but using 567 mg of 5-(1,2-benzoxazol-3-yl)-2,4-dimethoxybenzaldehyde, obtained in Step 1 of Example 8, 266 mg of 3,4-dimethylaniline, 601 mg of acetic acid and 1.86 g of molecular sieve 3A and then 276.5 of sodium cyanoborohydride in 12 ml of methanol for 20 hours at ambient temperature. After purification by flash chromatography on silica gel, elution being carried out with a mixture of cyclohexane and ethyl acetate (80/20 by volume), and then by crystallization from diisopropyl ether, 720 mg of [5-(1,2-benzisoxazol-3-yl)-2,4-dimethoxyphenyl]methyl-(3,4-dimethylphenyl)amine are obtained in the form of a white solid, the characteristics of which are as follows:

1H NMR spectrum at 400 MHz-δ in ppm-DMSO-D6: 2.03 (s, 3H); 2.06 (s, 3H); 3.87 (s, 3H); 3.99 (s, 3H); 4.19 (d, J=6.0 Hz, 2H); 5.74 (t, J=6.0 Hz, 1H); 6.26 (dd, J=2.5 and 8.0 Hz, 1H); 6.40 (d, J=2.5 Hz, 1H); 6.78 (d, J=8.0 Hz, 1H); 6.90 (s, 1H); 7.31 (t, J=8.0 Hz, 1H); 7.44 (s, 1H); 7.54 (d, J=8.0 Hz, 1H); 7.62 (t, J=8.0 Hz, 1H); 7.73 (d, J=8.0 Hz, 1H).

Mass spectrum (EI): m/z=388 (M+)

Step 2: The process is carried out as in Example 1, but using 680 mg of [5-(1,2-benzisoxazol-3-yl)-2,4-dimethoxyphenyl]methyl-(3,4-dimethylphenyl)-amine and 8.75 ml of a molar solution of boron tribromide in 12 ml of dichloromethane. Purification is carried out by flash chromatography on silica gel, elution being carried out with mixtures of cyclohexane and ethyl acetate (95/5 then 90/10 by volume).

By recovering the first fraction eluted and then crystallizing it from pentane, 22 mg of 4-(1,2-benzisoxazol-3-yl)-6-(3,4-dimethylphenylamino)methylbenzene-1,3-diol are obtained in the form of a yellow solid, the characteristics of which are as follows:

1H NMR spectrum at 400 MHz-δ in ppm-DMSO-D6: 2.04 (s, 3H); 2.07 (s, 3H); 4.13 (broad s, 2H); 5.65 (broad s, 1H); 6.31 (dd, J=3.0 and 8.5 Hz, 1H); 6.43 (d, J=3.0 Hz, 1H); 6.61 (s, 1H); 6.79 (d, J=8.5 Hz, 1H); 7.29 (t, J=8.0 Hz, 1H); 7.38 (s, 1H); 7.61 (t, J=8.0 Hz, 1H); 7.67 (d, J=8.0 Hz, 1H); 7.71 (d, J=8.0 Hz, 1H); from 9.60 to 10.2 (broad m, 2H)

Mass spectrum (EI): m/z=360 (M+)

By recovering the second fraction eluted and then crystallizing it from diisopropyl ether, 185 mg of 2-(1,2-benzisoxazol-3-yl)-4-(3,4-dimethylphenylamino)methyl-5-methoxyphenol are obtained in the form of a yellow solid, the characteristics of which are as follows:

1H NMR spectrum at 400 MHz-δ in ppm-DMSO-D6: 4.16 (d, J=5.5 Hz, 2H); 5.96 (broad t, J=5.5 Hz, 1H); 6.50 (broad t, J=7.5 Hz, 1H); 6.57 (d, J=8.5 Hz, 2H); 6.62 (s, 1H); 7.04 (dd, J=7.5 and 8.5 Hz, 2H); 7.29 (broad t, J=8.0 Hz, 1H); 7.39 (s, 1H); 7.61 (dt, J=1.0 and 8.0 Hz, 1H); 7.68 (d, J=8.0 Hz, 1H); 7.70 (d, J=8.0 Hz, 1H); from 9.65 to 10.2 (broad m, 2H)

Mass spectrum (EI): m/z=374 (M+)

### Examples 11 and 12

### 4-(1,2-Benzisoxazol-3-yl)-6-(3,4-dichlorophenylamino)methylbenzene-1,3-diol and 4-(1,2-benzisoxazol-3-yl)-6-(3,4-dichlorophenyl)iminomethylbenzene-1,3-diol

Step 1: The process is carried out as in Step 2 of Example 8, but using 567 mg of 5-(1,2-benzoxazol-3-yl)-2,4-dimethoxybenzaldehyde, obtained in Step 1 of Example 8, 356 mg of 3,4-dichlorolaniline, 601 mg of acetic acid and 1.86 g of molecular sieve 3A and then 276.5 of sodium cyanoborohydride in 12 ml of methanol for 20 hours at ambient temperature. After purification by flash chromatography on silica gel, elution being carried out with a mixture of cyclohexane and ethyl acetate (80/20 by volume), 800 mg of [5-(1,2-benzisoxazol-3-yl)-2,4-dimethoxyphenyl]methyl-(3,4-dichlorophenyl)-amine are obtained in the form of a white solid, the characteristics of which are as follows:

Mass spectrum (EI): m/z=429 (M+)

Step 2: The process is carried out as in Example 1, but using 4 g of [5-(1,2-benzisoxazol-3-yl)-2,4-dimethoxyphenyl]methyl-(3,4-dichlorophenyl)-amine and 78.3 ml of a molar solution of boron tribromide in 100 ml of dichloromethane. Purification is carried out by flash chromatography on silica gel, elution being carried out with mixtures of cyclohexane and ethyl acetate (95/5 then 90/10 by volume).

By recovering the first fraction eluted and then crystallizing it from pentane, 1.65 g of 4-(1,2-benzisoxazol-3-yl)-6-(3,4-dichlorophenylamino)methylbenzene-1,3-diol are obtained in the form of a white solid, the characteristics of which are as follows:

Mass spectrum (EI): m/z=401 (M+)

By recovering the second fraction eluted and then crystallizing it from diisopropyl ether, 465 mg of 4-(1,2-benzisoxazol-3-yl)-6-(3,4-dichlorophenyl)iminomethylbenzene-1,3-diol are obtained in the form of an ecru solid, the characteristics of which are as follows:

1H NMR spectrum at 400 MHz-δ in ppm-DMSO-D6: 6.62 (s, 1H); from 7.38 to 7.44 (m, 2H); from 7.65 to 7.71 (m, 2H); 7.73 (d, J=3.0 Hz, 1H); 7.79 (d, J=8.0 Hz, 1H); 7.88 (d, J=8.0 Hz, 1H); 7.92 (s, 1H); 9.00 (s, 1H); 11.15 (broad m, 1H); 13.2 (broad s, 1H)

Mass spectrum (EI): m/z=399 (M+)

### Example 13

### Pharmaceutical Composition

Tablets corresponding to the following formula were prepared:

Product of Example 2 . . . 0.2 g

Excipient for a tablet with a final mass of . . . 1 g (details of the excipient: lactose, talc, starch, magnesium stearate).

Biological Assay for Biologically Characterizing the Invention:

The inorganic phosphate released during the hydrolysis of ATP by the ATPase activity of Hsp82 is quantified by the malachite green method. In the presence of this reagent, formation of the inorganic phosphate-molybdate-malachite green complex occurs, which complex absorbs at a wavelength of 620 nm.

The products to be evaluated are incubated in a reaction volume of 30 μl, in the presence of 1 μM Hsp82 and of 250 μM of substrate (ATP) in a buffer composed of 50 mM Hepes-NaOH (pH 7.5), 1 mM DTT, 5 mM MgCl₂ and 50 mM KCl at 37° C. for 60 min. In parallel, a range of inorganic phosphate of between 1 and 40 μM is prepared in the same buffer. The ATPase activity is subsequently visualized by the addition of 60 μl of the biomol green reagent (Tebu). After incubation at ambient temperature for 20 min, the absorbance of the various wells is measured using a microplate reader at 620 nm. The concentration of inorganic phosphate of each sample is then calculated from the standard curve. The ATPase activity of Hsp82 is expressed as concentration of inorganic phosphate produced in 60 min. The effect of the various products tested is expressed as percentage inhibition of the ATPase activity.

The formation of ADP due to the ATPase activity of Hsp82 was used to develop another method for evaluating the enzymatic activity of this enzyme by application of an enzymatic coupling system involving pyruvate kinase (PK) and lactate dehydrogensase (LDH). In this kinetic-type spectrophotometric method, the PK catalyses the formation of ATP and of pyruvate from phosphoenol pyruvate (PEP) and from the ADP produced by Hsp82. The pyruvate formed, which is a substrate for LDH, is subsequently converted into lactate in the presence of NADH. In this case, the decrease in NADH concentration, measured by the decrease in absorbance at the wavelength of 340 nm, is proportional to the concentration of ADP produced by Hsp82.

The products tested are incubated in a reaction volume of 100 μl of buffer composed of 100 mM Hepes-NaOH (pH 7.5), 5 mM MgCl2, 1 mM DTT, 150 mM KCl, 0.3 mM NADH, 2.5 mM PEP and 250 μM ATP. This mixture is preincubated at 37° C. for 30 min before the addition of 3.77 units of LDH and 3.77 units of PK. The reaction is initiated by the addition of the product to be evaluated, at varying concentrations, and of Hsp82, at a concentration of 1 μM. The enzymatic activity of Hsp82 is then measured, continuously, in a microplate reader, at 37° C., at the wavelength of 340 nm. The initial rate of the reaction is obtained by measuring the slope of the tangent at the origin of the curve recorded. The enzymatic activity is expressed in μM of ADP formed per minute. The effect of the various products tested is expressed as percentage inhibition of the ATPase activity.

| Example | Structure | IC50 μM |
|---|---|---|
| 1 | 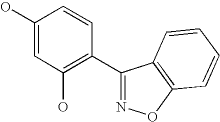 | B |
| 2 | 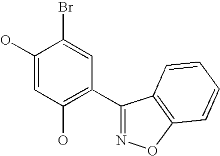 | B |
| 3 | 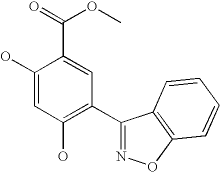 | C |
| 4 | 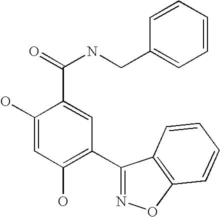 | C |
| 5 | 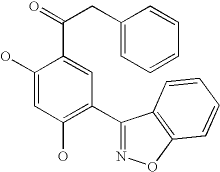 | C |
| 6 | 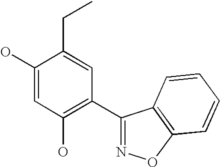 | B |
| 7 | 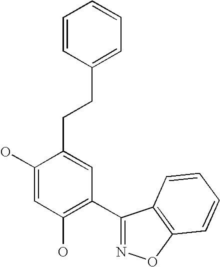 | B |
| 8 | 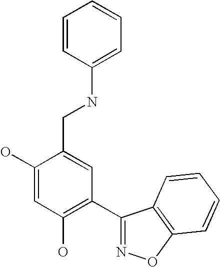 | B |
| 9 | 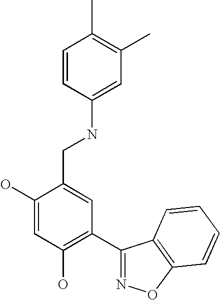 | A |
| 10 | 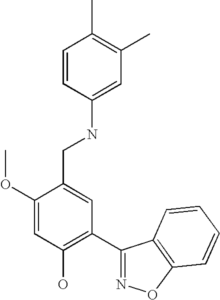 | C |
| 11 | 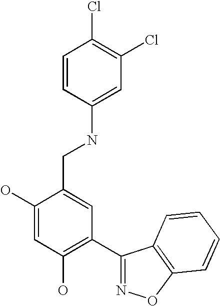 | B |
| 12 | 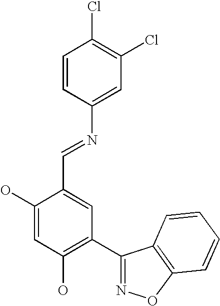 | B |

A: IC50 < 1 μM
B: 1 μM < IC50 < 10 μM
C: 10 μM < IC50 < 100 μM

What is claimed is:

1. A compound of formula (I): 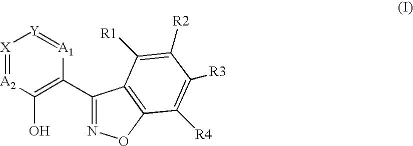
wherein:
A1 and A2, which may be identical or different, represent CH or N;
X represents CRa or N;
Y represents CRb or N;
R₁, R₂, R₃ and R₄, which may be identical or different, each a represent hydrogen or halogen atom, or a cyano, nitro, trifluoromethyl, OR₅, SR₅, NR₅R₆, C(O)R₅, C(O)OR₅, C(O)NR₅R₆, S(O)R₅, S(O)₂R₅, S(O)NR₅R₆, S(O)₂NR₅R₆, NR₆C(O)R₅, NR₆C(O)OR₅, alkyl, alkenyl, alkynyl, aryl, heteroaryl, aralkyl or heteroaralkyl radical; where the alkyl, alkenyl, alkynyl, aryl, heteroaryl, aralkyl or heteroaralkyl radicals can be substituted;
Rb represents a hydrogen or halogen atom, a cyano, nitro or trifluoromethyl radical or a (C₁-C₃ alkyl)n-W—(C₁-C₃ alkyl)m-R₅ radical;
Ra represents a halogen atom, a hydroxyl radical or a nitro radical when Rb is a hydrogen atom; Ra represents a hydrogen or a halogen atom, or a hydroxyl, methyl, ethyl, alkoxy, hydroxymethyl, nitro, carboxyl or cyano radical when Rb is other than a hydrogen atom;
R₅ and R₆, which may be identical or different, each represent a hydrogen atom or an alkyl, alkenyl, alkynyl, aryl, heteroaryl, aralkyl or heteroaralkyl radical; where the alkyl, alkenyl, alkynyl, aryl, heteroaryl, aralkyl or heteroaralkyl radicals can be substituted;
n and m are independently chosen from 0 and 1; and
W represents a single bond, an oxygen or sulphur atom, a C₁-C₃ alkyl, C₁-C₃ alkenyl, C₁-C₃ alkynyl, C₃-C₇ cycloalkyl, C₃-C₇ heterocycloalkyl, aryl, heteroaryl, NH, —CH═N—, N—(C₁-C₃ alkyl), C(O), C(O)—O, C(O)—NH, C(O)—N(C₁-C₃ alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃ alkyl), NH—S(O), NH—S(O)₂, N(C₁-C₃ alkyl)-S(O), N(C₁-C₃alkyl)-S(O)₂, O—S(O), O—S(O)₂, S(O)—O, S(O)₂—O, O—P(C₁-C₃ alkyl)(O), O—P(O)₂, NH—P(C₁-C₃ alkyl)(O), NH—P(O)₂, P(C₁-C₃ alkyl)(O)—NH or P(O)₂—NH radical; or
a racemate, enantiomer, diastereomer or prodrug of said compound or an inorganic or organic acid addition salt, or an inorganic or organic base addition salt of said compound, racemate, enantiomer, diastereomer or prodrug;
provided that the compound of formula (I) is other than 4-(1,2-benzisoxazol-3-yl)benzene-1,3-diol.

2. A compound according to claim 1 wherein:
A1 and A2, which may be identical or different, represent CH or N;
X represents CRa or N;
Y represents CRb or N;
R₁, R₂, R₃ and R₄, which may be identical or different, each represent a hydrogen or halogen atom, or a cyano, nitro, trifluoromethyl, OR₅, SR₅, NR₅R₆, C(O)R₅, C(O)OR₅, C(O)NR₅R₆, S(O)R₅, S(O)₂R₅, S(O)NR₅R₆, S(O)₂NR₅R₆, NR₆C(O)R₅, NR₆C(O)OR₅, alkyl, alkenyl, alkynyl, aryl, heteroaryl, aralkyl or heteroaralkyl radical; wherein the alkyl, alkenyl, alkynyl, aryl, heteroaryl, aralkyl or heteroaralkyl radicals can be substituted;
Rb represents a hydrogen or halogen atom, a cyano, nitro or trifluoromethyl radical or a (C₁-C₃ alkyl)n-W—(C₁-C₃ alkyl)m-R₅ radical;
Ra represents a halogen atom, a hydroxyl radical or a nitro radical when Rb is a hydrogen atom; Ra represents a hydrogen or a halogen atom, or a hydroxyl, methyl, ethyl, methoxy, hydroxymethyl, nitro, carboxyl or cyano radical when Rb is other than a hydrogen atom;
R₅ and R₆, which may be identical or different, each represent a hydrogen atom or an alkyl, alkenyl, alkynyl, aryl, heteroaryl, aralkyl or heteroaralkyl radical; wherein the alkyl, alkenyl, alkynyl, aryl, heteroaryl, aralkyl or heteroaralkyl radicals can be substituted;
n and m are independently chosen from 0 and 1; and
W represents a single bond, an oxygen or sulphur atom, a C₁-C₃ alkyl, C₁-C₃ alkenyl, C₁-C₃ alkynyl, C₃-C₇ cycloalkyl, C₃-C₇ heterocycloalkyl, aryl, heteroaryl, NH, N—(C₁-C₃ alkyl), C(O), C(O)—O, C(O)—NH, C(O)—N(C₁-C₃ alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃ alkyl), NH—S(O), NH—S(O)₂, N(C₁-C₃ alkyl)-S(O), N(C₁-C₃ alkyl)-S(O)₂, O—S(O), O—S(O)₂, S(O)—O, S(O)₂₋₀, O—P(C₁-C₃ alkyl)(O), O—P(O)₂, NH—P(C₁-C₃ alkyl)(O), NH—P(O)₂, P(C₁-C₃ alkyl)(O)—NH or P(O)₂—NH radical.

3. A compound according to claim 1 wherein:
A1 and A2, which may be identical or different, represent CH or N;
X represents CRa or N;
Y represents CRb or N;
R₁, R₂, R₃ and R₄, which may be identical or different, each represent a hydrogen or halogen atom, or a cyano, nitro, trifluoromethyl, OR₅, SR₅, NR₅R₆, C(O)R₅, C(O)OR₅, C(O)NR₅R₆, S(O)₂NR₅R₆, NR₆C(O)R₅, NR₆C(O)OR₅, alkyl, aryl, heteroaryl, aralkyl or heteroaralkyl radical; wherein the alkyl, alkenyl, alkynyl, aryl, heteroaryl, aralkyl or heteroaralkyl radicals are optionally substituted;
Ra represents a halogen atom, a hydroxyl or alkoxy radical or a nitro radical;
Rb represents a hydrogen or halogen atom or a (C₁-C₃ alkyl)n-W—(C₁-C₃ alkyl)m-R₅ radical;
R₅ and R₆, which may be identical or different, each represent a hydrogen atom or an alkyl, alkenyl, alkynyl, aryl, heteroaryl, aralkyl or heteroaralkyl radical; wherein the alkyl, alkenyl, alkynyl, aryl, heteroaryl, aralkyl or heteroaralkyl radicals are optionally substituted;
n and m are independently chosen from 0 and 1; and
W represents a single bond, an oxygen atom, a C₁-C₃ alkyl, C₂-C₃ alkenyl, C₃-C₇ heterocycloalkyl, aryl, heteroaryl, NH, —CH═N—, N—(C₁-C₃alkyl), C(O), C(O)—O, C(O)—NH, C(O)—N(C₁-C₃alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃alkyl), NH—S(O), NH—S(O)₂, N(C₁-C₃ alkyl)-S(O), N(C₁-C₃ alkyl)-S(O)₂, O—S(O), O—S(O)₂, S(O)—O or S(O)₂—O radical.

4. A compound according to claim 1 wherein:
A1 and A2, which may be identical or different, represent CH or N;
X represents CRa or N;
Y represents CRb or N;
R₁, R₂, R₃ and R₄, which may be identical or different, each represent a hydrogen or halogen atom, or a cyano, nitro, trifluoromethyl, OR₅, SR₅, NR₅R₆, C(O)R₅, C(O)OR₅, C(O)NR₅R₆, S(O)₂NR₅R₆, NR₆C(O)R₅, NR₆C(O)OR₅, alkyl, aryl, heteroaryl, aralkyl or heteroaralkyl radical; wherein the alkyl, alkenyl, alkynyl, aryl, heteroaryl, aralkyl or heteroaralkyl radicals are optionally substituted;
Ra represents a halogen atom, a hydroxyl radical or a nitro radical;
Rb represents a hydrogen or halogen atom or a (C₁-C₃ alkyl)n-W—(C₁-C₃ alkyl)m-R₅ radical;
R₅ and R₆, which may be identical or different, each represent a hydrogen atom or an alkyl, alkenyl, alkynyl, aryl, heteroaryl, aralkyl or heteroaralkyl radical; where the alkyl, alkenyl, alkynyl, aryl, heteroaryl, aralkyl or heteroaralkyl radicals are optionally substituted;
n and m are independently chosen from 0 and 1; and
W represents a single bond, an oxygen atom, a C₁-C₃ alkyl, C₂-C₃ alkenyl, C₃-C₇ heterocycloalkyl, aryl, heteroaryl, NH, N—(C₁-C₃ alkyl), C(O), C(O)—O, C(O)—NH, C(O)—N(C₁-C₃alkyl), O—C(O), O—C(O)—NH, NH—C(O), NH—C(O)—O, N(C₁-C₃ alkyl)-C(O), NH—O, N(C₁-C₃ alkyl)-O, S(O)—NH, S(O)₂—NH, S(O)—N(C₁-C₃ alkyl), S(O)₂—N(C₁-C₃alkyl), NH—S(O), NH—S(O)₂, N(C₁-C₃ alkyl)-S(O), N(C₁-C₃ alkyl)-S(O)₂, O—S(O), O—S(O)₂, S(O)—O or S(O)₂—O radical.

5. A compound according to claim 1, wherein;
A1 and A2, which may be identical or different, represent CH or N;
X represents CRa or N;
Y represents CRb or N;
R₁, R₂, R₃ and R₄, which may be identical or different, each represent a hydrogen or halogen atom or a —OR₅ radical;
Ra represents a halogen atom or a hydroxyl or alkoxy radical;
Rb represents a hydrogen or halogen atom or a (C₁-C₃ alkyl)n-W—R₅ radical;
R5 represents an alkyl, aralkyl, aryl or heteroaralkyl radical, all of which may be optionally substituted;
W represents a single bond, C(O), C(O)—O, C(O)—NH, NH, or —CH═N—; and
n represents 0 or 1.

6. A compound according to claim 1 wherein:
A1 and A2, which may be identical or different, represent CH or N;
X represents CRa or N;
Y represents CRb or N;
R₁, R₂, R₃ and R₄, which may be identical or different, each represent a hydrogen or halogen atom or a —OR₅ radical;
Ra represents a halogen atom or a hydroxyl radical;
Rb represents a hydrogen or halogen atom or a W—R₅ radical;
R₅ represents an alkyl, aralkyl or heteroaralkyl radical, each of which may be optionally substituted; and
W represents C(O), C(O)—O or C(O)—NH.

7. A compound according to claim 1 wherein A1 and A2 represent CH.

8. A compound according to claim 1 wherein X represents CRa, wherein Ra represents a hydroxyl radical.

9. A compound according to claim 1 wherein:
A1 and A2 represent CH;
X represents CRa, wherein Ra represents a hydroxyl or methoxy radical;
Y represents CRb wherein
Rb represents a hydrogen or bromine atom,
or Rb represents a —(CH₃)n-W—R5 radical wherein R5 represents an alkyl, phenyl or phenylalkyl radical, where the alkyl, phenyl or phenylalkyl radical is optionally substituted with one or more radicals chosen from halogen atoms and alkyl radicals;
R1, R2, R3 and R4 each represent a hydrogen atom;
W represents a single bond, C(O), C(O)—O, C(O)—NH, NH or —CH═N—; and
n represents 0 or 1.

10. A compound according to claim 1 wherein:
A1 and A2 represent CH;
X represents CRa wherein Ra represents a hydroxyl radical;
Y represents CRb wherein Rb represents a hydrogen or bromine atom, or wherein Rb represents a —W—R₅ radical wherein R₅ is chosen from optionally substituted alkyl and phenylalkyl radicals; and
R1, R2, R3 and R4 each represent a hydrogen atom.

11. A compound according to claim 1 wherein: Y represents CRb wherein Rb represents a hydrogen or bromine atom.

12. A compound according to claim 1 wherein:
A1 and A2 represent CH;
X represents CRa wherein Ra represents a hydroxyl radical;
Y represents CRb wherein Rb represents a hydrogen or bromine atom; and
R1, R2, R3 and R4 each represent a hydrogen atom.

13. A compound according to claim 1 which is:
4-(1,2-benzisoxazol-3-yl)-6-bromobenzene-1,3-diol;
5-(1,2-benzisoxazol-3-yl)-2,4-dihydroxybenzenecarboxylic acid methyl ester;
N1-phenylmethyl-5-(1,2-benzisoxazol-3-yl)-2,4-dihydroxybenzene-carboxamide;
1-{4-[(1,2-benzisoxazol-3-yl)-(1,3-dihydroxyphenyl]}-2-phenylethanone;
4-(1,2-benzisoxazol-3-yl)-6-ethylbenzene-1,3-diol;
4-(1,2-benzisoxazol-3-yl)-6-(2-phenylethyl)benzene-1,3-diol;
4-(1,2-benzisoxazol-3-yl)-6-(phenylamino)methylbenzene-1,3-diol;
4-(1,2-benzisoxazol-3-yl)-6-(3,4-dimethylphenylamino)methylbenzene-1,3-diol;
4-(1,2-benzisoxazol-3-yl)-6-(3,4-dichlorophenylamino)methylbenzene-1,3-diol; or
4-(1,2-benzisoxazol-3-yl)-6-(3,4-dichlorophenyl)iminomethylbenzene-1,3-diol; or
a racemate, enantiomer, diastereomer or prodrug of said compound or an inorganic or organic acid addition salt, or an inorganic or organic base addition salt of said compound, racemate, enantiomer, diastereomer or prodrug.

14. A compound according to claim 1 which is:
4-(1,2-benzisoxazol-3-yl)-6-bromobenzene-1,3-diol;
5-(1,2-benzisoxazol-3-yl)-2,4-dihydroxybenzenecarboxylic acid methyl ester;
N1-phenylmethyl-5-(1,2-benzisoxazol-3-yl)-2,4-dihydroxybenzene-carboxamide; or
1-{4-[(1,2-benzisoxazol-3-yl)-(1,3-dihydroxyphenyl]}-2-phenylethanone; or
a racemate, enantiomer, diastereomer or prodrug of said compound or an inorganic or organic acid addition salt, or an inorganic or organic base addition salt of said compound, racemate, enantiomer, diastereomer or prodrug.

15. A compound according to claim 1 which is:
4-(1,2-benzisoxazol-3-yl)-6-bromobenzene-1,3-diol; or
a racemate, enantiomer, diastereomer or prodrug of said compound or an inorganic or organic acid addition salt, or an inorganic or organic base addition salt of said compound, racemate, enantiomer, diastereomer or prodrug.

16. A compound according to claim 1 which is:
4-(1,2-benzisoxazol-3-yl)-6-(3,4-dimethylphenylamino)methylbenzene-1,3-diol; or
a racemate, enantiomer, diastereomer or prodrug of said compound or an inorganic or organic acid addition salt, or an inorganic or organic base addition salt of said compound, racemate, enantiomer, diastereomer or prodrug.

17. 4-(1,2-benzisoxazol-3-yl)benzene-1,3-diol, or a racemate, enantiomer, diastereomer or prodrug thereof, or an inorganic or organic acid addition salt, or an inorganic or organic base addition salt thereof, or of said racemate, enantiomer, diastereomer or prodrug.

18. A pharmaceutical composition comprising a pharmaceutically effective amount of a compound according to claim 1 and a pharmaceutically acceptable excipient.

19. A pharmaceutical composition comprising a pharmaceutically effective amount of a compound according to claim 13 and a pharmaceutically acceptable excipient.

20. A pharmaceutical composition comprising a pharmaceutically effective amount of a compound according to claim 17 and a pharmaceutically acceptable excipient.

21. A method of treating cancer comprising administering to a patient in need thereof, a pharmaceutically effective amount of a compound according to claim 1.

22. A method of treating cancer comprising administering to a patient in need thereof, a pharmaceutically effective amount of a compound according to claim 13.

23. A method of treating cancer comprising administering to a patient in need thereof, a pharmaceutically effective amount of a compound according to claim 17.

24. A pharmaceutical composition according to claim 18, further comprising at least one other cancer chemotherapeutic agent.

25. A pharmaceutical composition according to claim 19, further comprising at least one other cancer chemotherapeutic agent.

26. A pharmaceutical composition according to claim 20, further comprising at least one other cancer chemotherapeutic agent.

27. A method for preventing or treating a disease characterized by a disturbance in Hsp90 protein activity, in a patient in need thereof, comprising administering to such patient a pharmaceutically effective amount of a compound according to claim 1.

28. A method for preventing or treating a disease characterized by a disturbance in Hsp90 protein activity, in a patient in need thereof, comprising administering to such patient a pharmaceutically effective amount of a compound according to claim 13.

29. A method for preventing or treating a disease characterized by a disturbance in Hsp90 protein activity, in a patient in need thereof, comprising administering to such patient a pharmaceutically effective amount of a compound according to claim 17.

30. A method of inhibiting Hsp90 protein activity, in a patient in need thereof, comprising administering to such patient a pharmaceutically effective amount of a compound according to claim 1.

31. A method of inhibiting Hsp90 protein activity, in a patient in need thereof, comprising administering to such patient a pharmaceutically effective amount of a compound according to claim 13.

32. A method of inhibiting Hsp90 protein activity, in a patient in need thereof, comprising administering to such patient a pharmaceutically effective amount of a compound according to claim 17.

33. A method for treating solid or liquid tumor cancer, in a patient in need thereof, comprising administering to such patient a pharmaceutically effective amount of a compound according to claim 1.

34. A method for treating solid or liquid tumor cancer, in a patient in need thereof, comprising administering to such patient a pharmaceutically effective amount of a compound according to claim 13.

35. A method for treating solid or liquid tumor cancer, in a patient in need thereof, comprising administering to such patient a pharmaceutically effective amount of a compound according to claim 17.

36. A method for treating a cancer resistant to cytotoxic agents, in a patient in need thereof, comprising administering to such patient a compound according to claim 1.

37. A method for treating a cancer resistant to cytotoxic agents, in a patient in need thereof, comprising administering to such patient a compound according to claim 13.

38. A method for treating a cancer resistant to cytotoxic agents, in a patient in need thereof, comprising administering to such patient a compound according to claim 17.

39. A method according to claim 21 wherein the cancer is selected from lung cancer, breast cancer, ovarian cancer, glioblastomas, chronic myeloid leukaemias, acute lymphoblastic leukaemias, prostate cancer, pancreatic cancer, colon cancer, metastatic melanomas, thyroid tumours and renal carcinomas.

40. A method according to claim 22 wherein the cancer is selected from lung cancer, breast cancer, ovarian cancer, glioblastomas, chronic myeloid leukaemias, acute lymphoblastic leukaemias, prostate cancer, pancreatic cancer, colon cancer, metastatic melanomas, thyroid tumours and renal carcinomas.

41. A method according to claim 23 wherein the cancer is selected from lung cancer, breast cancer, ovarian cancer, glioblastomas, chronic myeloid leukaemias, acute lymphoblastic leukaemias, prostate cancer, pancreatic cancer, colon cancer, metastatic melanomas, thyroid tumours and renal carcinomas.

42. A method of treating cancer, in a patient in need thereof, comprising administering to such patient a compound according to claim 1, in combination with at least one other cancer chemotherapeutic agent.

43. A method of treating cancer, in a patient in need thereof, comprising administering to such patient a compound according to claim 13, in combination with at least one other cancer chemotherapeutic agent.

44. A method of treating cancer, in a patient in need thereof, comprising administering to such patient a compound according to claim 17, in combination with at least one other cancer chemotherapeutic agent.

45. A method of treating cancer, in a patient in need thereof, comprising administering to such patient a compound according to claim 1, in combination with radiotherapy.

46. A method of treating cancer, in a patient in need thereof, comprising administering to such patient a compound according to claim 13, in combination with radiotherapy.

47. A method of treating cancer, in a patient in need thereof, comprising administering to such patient a compound according to claim 17, in combination with radiotherapy.
